(19) United States
(12) Patent Application Publication
(10) Pub. No.: US 2026/0151232 A1
(43) Pub. Date: Jun. 4, 2026

(54) Tissue Fixation Device With Improved Tissue Securement

(71) Applicant: Evalve, Inc., Santa Clara, CA (US)

(72) Inventors: Chad J. Abunassar, Alameda, CA (US); Grant Glaze, San Francisco, CA (US); Thomas R. Yssels, Mountain View, CA (US); Clara Park, Santa Clara, CA (US)

(73) Assignee: Evalve, Inc., Santa Clara, CA (US)

(21) Appl. No.: 19/460,493

(22) Filed: Jan. 27, 2026

Related U.S. Application Data
(63) Continuation of application No. PCT/US2025/031404, filed on May 29, 2025.
(60) Provisional application No. 63/654,239, filed on May 31, 2024.

(51) Int. Cl. A61F 2/24

(52) U.S. Cl. CPC A61F 2/2463; A61F 2220/0016; A61F 2230/0045

(57) ABSTRACT

A fixation device includes a first proximal element and a first distal element. The first proximal element has a gridded frame disposed between a first end portion and a second end portion and a plurality of frictional elements. The gridded frame has a plurality of axial struts and a plurality of transverse struts intersecting the axial struts to define a plurality of openings. At least some of the frictional elements extend from at least some of the transverse struts. The first distal element is disposed in opposition to the first proximal element and has a cavity disposed between a first end portion and a second end portion thereof. The cavity is configured to receive at least a portion of the gridded frame.

### CROSS-REFERENCE TO RELATED APPLICATIONS

This application is a continuation of International Application No. PCT/US 2025/031404, filed May 29, 2025, which claims the benefit of U.S. Provisional Application No. 63/654,239, filed May 31, 2024, the disclosures of which are hereby incorporated by reference in their entireties.

### BACKGROUND

The cardiac cycle is divided into two phases—diastole and systole. Diastole is generally characterized by the muscular relaxation of the heart and the filling of its chambers with blood. On the other hand, systole is generally characterized by the muscular contraction of the ventricles which pumps blood from the ventricles to the arteries. During ventricular systole, ventricular pressure increases relative to atrial pressure resulting in the closure of the mitral valve and the tricuspid valve. The mitral valve separates the left atrium from the left ventricle, and the tricuspid valve separates the right atrium from the right ventricle. These valves operate as check valves preventing blood from flowing back into the atria during ventricular contraction. However, valvular insufficiency may appear in one or both of these valves which may result in a regurgitative flow back into the atrium across the effected valve. Such regurgitative flow can be in the form of mitral valve regurgitation (“MVR”) and/or tricuspid valve regurgitation (“TVR”). Left untreated, MVR and TVR can lead to severe health consequences, such as progressive heart failure, cardiac arrythmias, pulmonary hypertension, stroke, and endocarditis, to name a few.

MVR and TVR can have a variety of etiologies which typically fall into the categories of degenerative (primary) and functional (secondary) regurgitation. Degenerative valve regurgitation principally occurs due to abnormalities or degeneration of the valve apparatus, such as the valve leaflets, valve annulus, chordae tendineae, and/or papillary muscles. One example of a degenerative valve condition is mitral valve prolapse. Functional valve regurgitation is often a secondary condition that arises from underlying heart conditions or diseases that affect the structure or function of the heart. Examples of conditions that can result in functional regurgitation include dilated cardiomyopathy, ischemic heart disease, pulmonary hypertension, and heart failure. Regardless of the underlying condition precipitating the regurgitative flow, the primary mechanism by which regurgitation occurs is the failure of the valve leaflets to properly and completely seal or coapt during systole which allows a jet of blood to flow back into the atrium between the effected leaflets.

Treatment options for MVR and TVR generally include Guideline-Directed Medical Therapy (“GDMT”), valve replacement, and valve repair. GDMT usually involves the administration of a combination of drugs that treat an underlying heart condition. Valve replacement and repair may include open-heart surgical options and catheter-based options. Catheter-based repair procedures are sometimes referred to as transcatheter edge-to-edge repair (“TEER”).

### BRIEF SUMMARY OF THE DISCLOSURE

In one aspect of the disclosure, a fixation device may include a first proximal element which may have a first end portion, a second end portion, a gridded frame disposed between the first end portion and the second end portion, and a plurality of frictional elements. The gridded frame may have a plurality of axial struts and a plurality of transverse struts intersecting the axial struts to define a plurality of openings extending through the first proximal element. At least some of the frictional elements may extend from at least some of the transverse struts. The fixation device may also include a first distal element disposed in opposition to the first proximal element. The first distal element may have a first end portion, a second end portion, and a cavity disposed between the first end portion and the second end portion. The cavity may be configured to be at least partially receive the gridded frame of the first proximal element. The first proximal element may be moveable relative to the first distal element between a first position and a second position, and, when the first proximal element is in the second position, the gridded frame may nest within the cavity of the first distal element.

Additionally, the first distal element may include a strutted frame disposed between the first end portion and the second end portion of the first distal element. The strutted frame may have a pair of inner axial struts and a pair of outer axial struts. The plurality of frictional elements may include a pair of inner frictional elements and a pair of outer frictional elements arranged in a row. When the first proximal element is in the second position, the inner axial struts may be disposed between a corresponding pair of inner and outer frictional elements. The inner and outer axial struts may define a plurality of elongate openings disposed therebetween. The outer axial struts may each have a proximal surface defining a reference plane, and the inner axial struts may be offset from the reference plane in a distal direction.

Also, the first proximal element may include an elongate arm that may extend from the first end portion to the second end portion, and the gridded frame may extend outwardly from the elongate arm such that the elongate arm has a first width, and the gridded frame has a second width greater than the first width. The axial struts of the first proximal element may define lateral boundaries of the gridded frame, and the transverse struts of the first proximal element may extend from the elongate arm to respective axial struts of the first proximal element. The outer axial struts of the first distal element may at least partially define the cavity and a third width greater than the second width. Furthermore, the elongate arm may have opposed side edges. The side edges may form at least some of the plurality of frictional elements, and the transverse struts may extend from a corresponding side edge of the elongate arm.

Further, the first proximal element may define a longitudinal axis extending between the first end portion and the second end portion. The inner and outer frictional elements may be angled one of inwardly toward the longitudinal axis and outwardly away from the longitudinal axis such that the outer frictional elements form a first tilt angle and the inner frictional elements form a second tilt angle relative to a distal surface of the first proximal element. The first tilt angle and the second tilt angle may be equal. Alternatively, the first tilt angle may be greater than the second title angle. Also, the plurality of frictional elements may be curved in a direction toward the first end portion of the first proximal element.

Additionally, the fixation device may further include a gripping device. The gripping device may have a first bend feature and may include the first proximal element. The first end portion of the first proximal element may be coupled to a first bend feature and may be configured to flex to allow the first proximal element to move relative to the first distal element between the first and second positions thereof. The gripping device may further include a base section, and the first bend feature may be coupled to the base section. The base section may be coupled to one of a center of the fixation device and the first distal element.

Also, the plurality of frictional elements may include a first frictional element and a second frictional element offset from the first frictional element in a longitudinal direction. The first frictional element may be angled relative to a distal surface of the first proximal element by a first angle, and the second frictional element being angled relative to the distal surface of the first proximal element by a second angle. The first angle may differ from the first angle. The first angle may be greater than the second angle, and the first frictional element may be closer to the first end portion than the second end portion. The first end portion may be a fixed end of the first proximal element, and the second end portion may be a free end of the first proximal element. The second end portion may be bent proximally in a direction away from the frictional elements and may define an opening configured to receive a proximal element line for actuation of the first proximal element to between the first and second positions thereof.

Further, the first proximal element may define a longitudinal axis extending between the first end portion and the second end portion thereof. The first proximal element may be bent in a transverse plane which may extend in a direction transverse to the longitudinal axis. In this regard, the first proximal element may be bent proximally such that a proximal surface thereof is concave in the transverse plane, and a distal surface of the first proximal element is convex in the transverse plane. The plurality of frictional elements may include a pair of inner frictional elements and a pair of outer frictional elements each arranged in a row, and the outer frictional elements may extend in a direction away from the inner frictional elements. Alternatively, the first proximal element may be bent distally such that a proximal surface thereof is convex in the transverse plane, and a distal surface of the first proximal element is concave in the transverse plane. The plurality of frictional elements may include a pair of inner frictional elements and a pair of outer frictional elements each arranged in a row. The outer frictional elements may extend in a direction toward the inner frictional elements.

Additionally, the plurality of frictional elements may be arranged in a plurality of rows of frictional elements such that at least two rows of the frictional elements each include four frictional elements. The first proximal element may define a length extending between the first end portion and the second end portion. The length may be bisected by a midline, and the at least two rows of frictional elements that each have four frictional elements may be disposed at opposite sides of the midline.

Also, the plurality of frictional elements may be arranged in six rows of frictional elements distributed along a length of the first proximal element. The first row of frictional elements may be closest to the first end portion of the first proximal element, and the sixth row of frictional elements may be closest to the second end portion of the first proximal element. The first and sixth rows of frictional elements may have fewer frictional elements than every row of frictional elements therebetween. Further, a second and fifth row of frictional elements may each include three frictional elements, and a third and fourth row of frictional elements may each include four frictional elements. Alternatively, the sixth row of frictional elements may have less friction elements than the other rows of frictional elements.

Further, the fixation device may further include a second proximal element which may include a first end portion, a second end portion, a gridded frame disposed between the first end portion and the second end portion, and a plurality of frictional elements. The gridded frame of the second proximal element may have a plurality of axial struts and a plurality of transverse struts intersecting the axial struts to define a plurality of openings extending through the second proximal element. At least some of the frictional elements of the second proximal element may extend from at least some of the transverse struts of the second proximal element. Also, a second distal element may be disposed in opposition to the second proximal element and may have a first end portion, a second end portion, and a cavity disposed between the first end portion and the second end portion of the second distal element. The cavity may be configured to receive the gridded frame of the second proximal element.

In another aspect of the present disclosure, a fixation device for fixation of tissue may include a center portion, a first distal element, and a gripping device. The first distal element may extend outwardly relative to the center portion and may be pivotable relative thereto between an opening position and a closed position. The gripping device may include a base section, a first bend feature, and a first proximal element. The first proximal element may be disposed in opposition to the first distal element and may be moveable relative thereto to capture tissue disposed between the first distal element and the first proximal element. The first proximal element may have an elongate arm and a plurality of frictional elements extending from the elongate arm. The elongate arm may have a first end portion, a second end portion, and a longitudinal axis extending between the first end portion and the second end portion. The elongate arm may define a first curvature curved in a plane parallel to the longitudinal axis. The first bend feature may be coupled to the base section and the first end portion of the elongate arm. The first bend feature may be configured to flex to allow the first proximal element to move relative to the first distal element.

Additionally, the base section may be coupled to the center portion of the fixation device. Alternatively, the base section may be coupled to the first distal element. The elongate arm may include a distal surface and a proximal surface. The distal surface may face the first distal element, and the frictional elements may extend from the distal surface. The distal surface may be concavely curved at the first curvature, and the proximal surface may be convexly curved at the first curvature.

Also, the elongate arm may define a second curvature curved in the plane parallel to the longitudinal axis. The distal surface may be convexly curved at the second curvature, and the proximal surface may be concavely curved at the second curvature. The first curvature may be closer to the first end portion of the elongate arm than the second curvature. The first curvature may define an apex located at a midpoint between the first end portion and second end portion. The first curvature may define a first center of curvature located distal of the distal surface. Also, the second curvature may define a second center of curvature located proximal of the proximal surface.

Further, the proximal element may further include a first loop and a second loop each extending from the proximal surface. Each loop may be configured to receive a proximal element line for actuation of the proximal element. The first loop may be located at a midpoint of the elongate arm between the first and second end portions, and the second loop may be located at the second end portion.

Additionally, the fixation device may further include a second distal element extending outwardly relative to the center portion and may be pivotable relative thereto between an opening position and a closed position. The fixation device may also include a second proximal element disposed in opposition to the second distal element and may be moveable relative thereto to capture tissue disposed between the second distal element and the second proximal element. The second proximal element may have an elongate arm and a plurality of frictional elements extending from the elongate arm of the second proximal element. The elongate arm of the second proximal element may have a first end portion, a second end portion, and a longitudinal axis extending between the first end portion and second end portion of the second proximal element. The elongate arm of the second proximal element may define a first curvature curved in the plane parallel to the longitudinal axis. Also, the fixation device may further include a second bend feature coupled to the base section and the first end portion of the second proximal element. The second bend feature may be configured to flex to allow the second proximal element to move relative to the second distal element.

In another aspect of the present disclosure, a fixation device for fixation of leaflets of a heart valve may include a first distal element, a second distal element, a first proximal element, and a second proximal element. The first proximal element may be moveable relative to the first distal element to grasp a first leaflet disposed therebetween. The first proximal element may have a proximal side and a distal side. The second proximal element may be moveable relative to the second distal element to grasp a second leaflet disposed therebetween. The second proximal element may have a proximal side and a distal side. Additionally, the fixation device may include a first frictional element connected to the first proximal element. The first frictional element may be moveable from a first position to a second position by the presence of the first leaflet between the first distal element and the first proximal element. In the first position, a first portion of the first frictional element may extend from the proximal side of the first proximal element and, and in the second position, the first portion of the first frictional element may extend from the distal side of the first proximal element to frictionally engage the first leaflet. The fixation device may also include a second frictional element connected to the second proximal element. The second frictional element may be moveable from a first position to a second position by the presence of the second leaflet between the second distal element and the first proximal element. In the first position, a first portion of the second frictional element may extend from the proximal side of the second proximal element, and in the second position, the second frictional element may extend from the distal side of the second proximal element to frictionally engage the second leaflet.

Additionally, the first and second frictional elements may be biased toward their respective first position. The proximal side of the first proximal element may be defined by a proximal surface, the distal side of the first proximal element may be defined by a distal surface. The first portion of the first frictional element may be configured to pass through the proximal and distal surfaces of the first proximal element when transitioning from the first position to the second position. Further, the proximal side of the second proximal element may be defined by a proximal surface, and the distal side of the second proximal element may be defined by a distal surface. The first portion of the second frictional element may be configured to pass through the proximal and distal surfaces of the second proximal element when transitioning from the first position to the second position.

Also, the first frictional element may include a second portion. The second portion may extend from the distal side of the first proximal element when in the first position and may extend from the proximal side of the first proximal element when in the second position. Further, the second frictional element may include a second portion. The second portion of the second frictional element may extend from the distal side of the second proximal element when in the first position and may extend from the proximal side of the second proximal element when in the second position. The first and second frictional elements may each be connected to the first and second proximal elements, respectively, via a hinge. The hinge may be a living hinge, or may be a modular hinge, such as a hinge pin. The hinge may be disposed between the first and second portions of the respective first and second frictional elements.

Further, the first portion of the first frictional element may include a prong, and when the first proximal element is in the first position, the prong may be disposed one of proximal to the proximal side of the first proximal element and between the proximal side and the distal side of the first proximal element. Also, the first portion of the second frictional element may include a prong, and when the second proximal element is in the first position, the prong of the second frictional element may be disposed one of proximal to the proximal side of the second proximal element and between the proximal side and the distal side of the second proximal element. The first portion of the first and second frictional elements may be in the shape of a hook. The first and second frictional elements may each include a material visible under fluoroscopy. The material may be nitinol, platinum-iridium, and/or tantulum.

### BRIEF DESCRIPTION OF THE DRAWINGS

FIG. 1A is a cross-sectional representation of a heart illustrating its four valves.

FIG. 1B is a cross-sectional representation of a heart illustrating the left ventricle and left atrium during systole.

FIG. 2A is a schematic view of a mitral valve during normal coaptation.

FIG. 2B is a schematic view of a mitral valve during regurgitate coaptation.

FIGS. 3A and 3B are schematic views of a fixation device according to an embodiment of the present disclosure grasping leaflets of a mitral valve.

FIG. 4A is a perspective view of a fixation device according to another embodiment of the present disclosure.

FIG. 4B is a perspective view of the fixation device of FIG. 4A including a covering.

FIG. 5A is a perspective view of a gripping device of the of the fixation device of FIG. 4A according to an embodiment of the present disclosure.

FIG. 5B is an elevational view of the gripping device of FIG. 5A.

FIG. 6A is a perspective view of a gripping device according to another embodiment of the present disclosure.

FIG. 6B is a partial schematic view of the gripping device of FIG. 6A coupled to a distal element of the fixation device of FIG. 4A.

FIG. 6C is a partial schematic view of a gripping device according to an alternative embodiment of the present disclosure coupled to a distal element according to an alternative embodiment of the present disclosure.

FIG. 7A is an elevational view of a coupling system according to an embodiment of the present disclosure for coupling the fixation device of FIG. 4A and a delivery system.

FIGS. 7B and 7C are schematic views of the coupling system of FIG. 7A in respective first and second configurations.

FIGS. 8A and 8B are schematic cross-sectional views of a coupling system according to another embodiment of the present disclosure for coupling a fixation device, such as the fixation device of FIG. 4A, and a delivery system.

FIGS. 9A-9B, 10A-10B, 11A-11B, 12A-12B and 13A-13C illustrate the fixation device of FIG. 4A in various possible positions during introduction and placement of the device within a mammalian body to perform a therapeutic procedure.

FIG. 14 is a perspective view of the fixation device of FIG. 4A including a locking mechanism according to an embodiment of the present disclosure and illustrating a plurality of proximal element lines and a lock line coupled to the fixation device.

FIG. 15 is an elevational view of the locking mechanism and proximal elements of the fixation device of FIG. 14 and illustrating a lock line and single proximal element line respectively coupled thereto.

FIG. 16 is a schematic view of the fixation device of FIG. 4A coupled to a delivery system and illustrating a plurality of proximal element lines coupled to a shaft of the delivery system.

FIGS. 17A and 17B are partial enlarged views of a distal end portion of the delivery system shaft of FIG. 16 according to an embodiment of the present disclosure.

FIG. 17C is a cross-sectional view of the delivery system shaft taken along line C-C of FIG. 17B.

FIG. 17D is a partial perspective view of a distal end portion of one of the proximal element lines of FIG. 16 including a catch element according to an embodiment of the present disclosure.

FIG. 17E is a partial elevational view of the delivery system shaft of FIG. 17A having holes configured to receive the catch element of FIG. 17D.

FIG. 17F is a partial elevational view of the delivery system shaft of FIG. 17A and an actuator rod disposed therein intersecting the holes of the delivery system shaft.

FIG. 17G is a partial elevational view of a distal end portion of one of the proximal element lines of FIG. 16 including a catch element according to another embodiment of the present disclosure.

FIG. 18A is an enlarged cross-sectional view of the locking mechanism of FIG. 14 taken along a midline thereof and in an unlocked configuration.

FIG. 18B is an enlarged elevational view of the locking mechanism of FIG. 14 and in a locked configuration.

FIG. 18C is a perspective view of a release harness of the locking mechanism of FIG. 14.

FIG. 19A is an elevational view of a locking mechanism of the fixation device of FIG. 4A according to another embodiment of the present disclosure.

FIG. 19B is a transparent perspective view of a binding plate of the locking mechanism of FIG. 19A.

FIG. 19C is an enlarged elevational view of the locking mechanism of FIG. 19A.

FIG. 20A is a perspective view of a fixation device according to a further embodiment of the present disclosure.

FIGS. 20B and 20C are perspective views of a gripping device of the fixation device of FIG. 20A.

FIG. 20D is a perspective view of a distal element of the fixation device of FIG. 20A.

FIG. 20E cross-sectional view of the distal element of FIG. 20D.

FIGS. 20F and 20G are partial perspective views of the gripping device of FIG. 20B interacting with the distal element of FIG. 20D.

FIG. 20H is a cross-sectional view of the gripping device of FIG. 20B and the distal element of FIG. 20D interacting with a leaflet.

FIG. 21 is a schematic cross-sectional view of a proximal element according to another embodiment of the present disclosure.

FIG. 22 is a schematic cross-sectional view of a proximal element according to a further embodiment of the present disclosure.

FIG. 23 is a schematic side view of a proximal element according to another embodiment of the present disclosure.

FIG. 24 is a schematic side view of a proximal element according to a further embodiment of the present disclosure.

FIG. 25 is a schematic side view of a proximal element according to another embodiment of the present disclosure.

FIG. 26 is a schematic bottom view of a proximal element according to a further embodiment of the present disclosure.

FIG. 27 is a schematic bottom view of a proximal element according to another embodiment of the present disclosure.

FIG. 28A is schematic bottom view of a proximal element according to a further embodiment of the present disclosure.

FIG. 28B is a schematic end view of the proximal element of FIG. 28A.

FIG. 29A are schematic views of a proximal element according to another embodiment of the present disclosure.

FIG. 29B is a schematic end view of the proximal element of FIG. 29A.

FIG. 30A is a perspective view of a gripping device according to a further embodiment of the present disclosure.

FIG. 30B is an elevational view of the gripping device of FIG. 30A.

FIG. 31A is a perspective view of a gripping device according to another embodiment of the present disclosure.

FIG. 31B is an elevational view of the gripping device of FIG. 31A.

FIG. 32A is a perspective view of a gripping device according to another embodiment of the present disclosure.

FIG. 32B is another perspective view of the gripping device of FIG. 32A.

FIG. 33A is a schematic view of a proximal element according to a further embodiment of the present disclosure.

FIG. 33B is a schematic side view of the proximal element of FIG. 33A.

FIGS. 33C and 33D illustrate the proximal element of FIG. 33A interacting with a leaflet.

FIG. 34 is a schematic partial top view of a proximal element according to another embodiment of the present disclosure.

FIG. 35A is an elevational view of a gripping device according to another embodiment of the present disclosure and including proximal elements in an exemplary relaxed configuration.

FIG. 35B is a partial view of the gripping device of FIG. 35A with a gripper thereof in an exemplary first grasping configuration.

FIG. 35C is a partial transparent view of the gripping device of FIG. 35A with a gripper thereof in an exemplary second grasping configuration and in relation to a fixation element.

### DETAILED DESCRIPTION

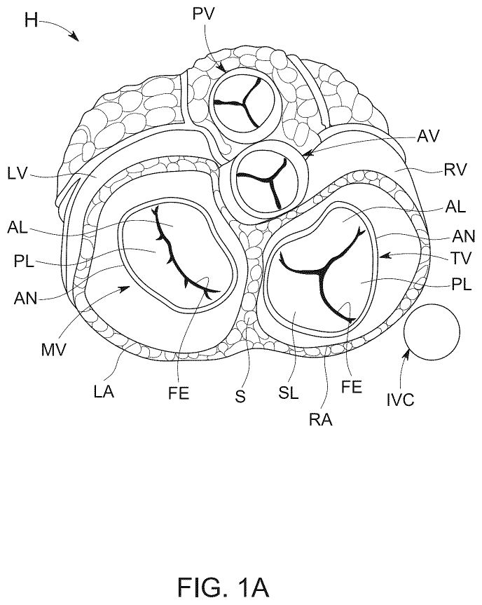

The valves of a normal heart H are illustrated in FIGS. 1A and 1B. These valves include the mitral valve MV, the tricuspid valve TV, the aortic valve AV, and the pulmonary valve PV. The mitral valve MV separates the left atrium LA and the left ventricle LV, and the tricuspid valve TV separates the right atrium RA and the right ventricle RV. The mitral valve MV and the tricuspid valve TV are sometimes referred to as the atrioventricular valves. The mitral valve MV is a bicuspid valve in that it has two leaflets referred to as the posterior leaflet PL and the anterior leaflet AL. The tricuspid valve TV typically has three leaflets referred to as the anterior leaflet AL, the posterior leaflet PL, and the septal leaflet SL. However, studies have shown that, although the TV is typically composed of three leaflets of unequal size, in many cases, two or more than three leaflets may be present as anatomic variants in healthy subjects. Thus, reference herein to the tricuspid valve TV should be understood to refer to the atrioventricular valve located between the right atrium RA and right ventricle RV regardless of the number of leaflets be it two, three, or more than three leaflets. However, exemplary embodiments discussed herein refer to the usual anatomic structure of the tricuspid valve TV that includes three leaflets.

As illustrated in FIG. 1B, the anterior leaflet AL and posterior leaflet PL of the mitral valve MV extend from a valve annulus AN to respective free edges FE. The free edges FE are secured to the lower portions of the left ventricle LV through chordae tendineae CT (referred to hereinafter as the chordae). The chordae CT include a plurality of branching tendons that are attached to papillary muscles PM at the lower portions of the left ventricle LV and extend upwardly to the lower surfaces of each of the valve leaflets where they are attached. The three leaflets of the tricuspid valve TV similarly extend from a valve annulus AN to respective free edges FE which are secured via chordae to the papillary muscles of the right ventricle RV.

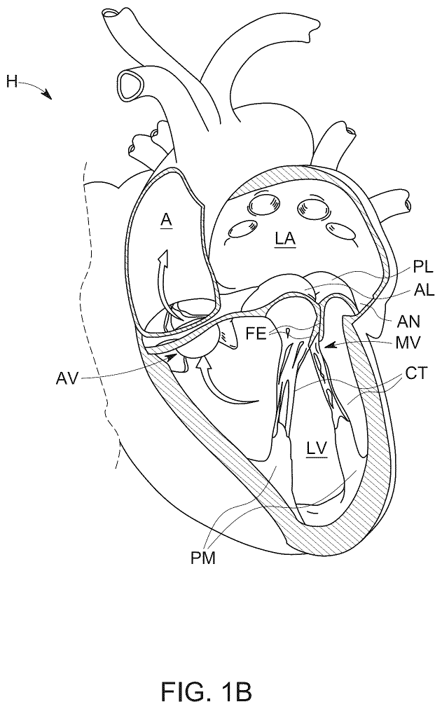

The mitral valve MV depicted in FIGS. 1B and 2A illustrate the proper functioning of an atrioventricular valve during ventricular systole. As the ventricles contract, the free edges FE of adjacent leaflets LF meet along a line of coaptation LOC. The joinder of the leaflets LF at this line of coaptation LOC seals off the ventricle from the atrium and prevents the back flow of blood or “regurgitation” from entering into the atrium. Thus, with the right atrium RA and left atrium LA respectively sealed off by the mitral valve MV and tricuspid valve TV, blood in the left ventricle LV can only flow through the aortic valve AV to the body, and blood in the right ventricle RV can only flow through the pulmonary valve PV to the lungs.

A number of structural defects in the heart H can cause mitral valve regurgitation (“MVR”) and/or tricuspid valve regurgitation (“TVR”). MVR and TVR occur when their respective leaflets LF do not close properly allowing leakage from the ventricle into the atrium. The mitral valve MV depicted in FIG. 2B illustrates valvular insufficiency of an atrioventricular valve resulting in regurgitation. In the depicted example, an enlargement of the heart H may cause the valve annulus AN to become enlarged, making it impossible for the free edges FE of the valve leaflets LF to meet during systole. This may result in a gap G between the leaflets LF which allows blood to leak through the valve. In another example, ruptured or elongated chordae CT can cause a valve leaflet LF to prolapse at least due to inadequate tension transmitted to the leaflet via the chordae CT. While an adjacent leaflet LF may maintain a normal profile, the prolapsing leaflets LF may flail about preventing the proper joinder between the leaflets LF resulting in leakage into the atrium. In a further example, regurgitation can occur in patients who have suffered ischemic heart disease which may result in weak ventricular contractions insufficient to effect proper closure.

The present disclosure describes exemplary systems, devices, and methods for percutaneously repairing a valve to treat cardiac valve regurgitation, particularly MVR and TVR. When referring to such disclosed systems, devices, and methods, the term “proximal” (P) shall mean closer to the user or in a direction toward a device to be manipulated by the user outside the patient's body, and the term “distal” (D) shall mean more distant from the user or in a direction toward a device that is positioned at the treatment site within the patient's body (e.g., fixation device 112). With respect to the mitral valve and tricuspid valve, “proximal” shall refer to the atrial or upstream side of the valve leaflets, and “distal” shall refer to the ventricular or downstream side of the valve leaflets.

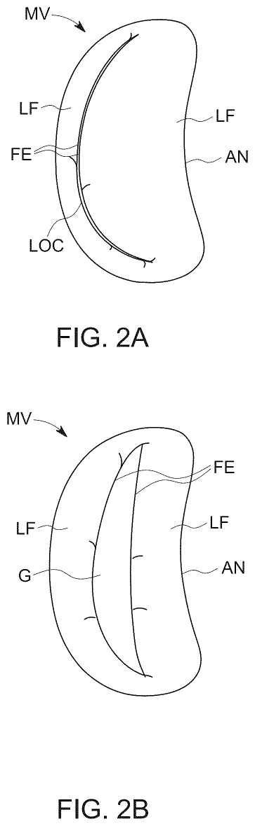

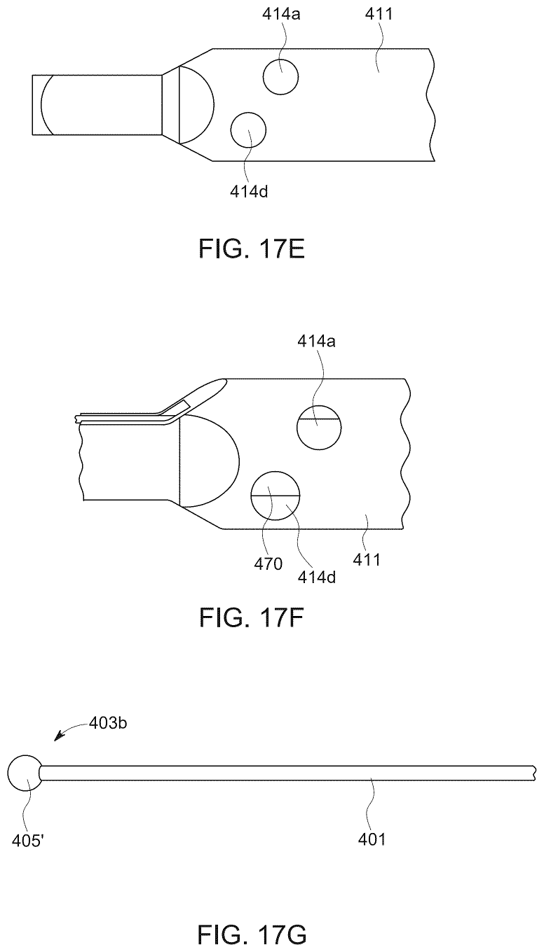

FIGS. 3A and 3B depict a fixation device 12, according to an embodiment of the present disclosure, grasping leaflets LF of an atrioventricular valve, which is illustrated as a mitral valve MV. Fixation device 12 may be releasably coupled to a distal end of a shaft 11 of a delivery system 600 (see FIG. 16) to form an interventional tool 10. Fixation device 12 may include distal elements 20 (also referred to herein as fixation elements) and proximal elements 40 (also referred to herein as gripping elements). Distal and proximal elements 20, 40 may be moveable relative to each other and may protrude radially outward relative to a longitudinal axis A1 of fixation device 12. As shown in FIG. 3A, fixation device 12 may be positionable on opposite sides of adjacent leaflets LF of the valve so as to capture or retain the leaflets LF therebetween. In this regard, proximal elements 40 may be positioned at a proximal side of the valve leaflets LF, and distal elements 20 may be positioned on a distal side of the valve leaflets LF. Proximal elements 40 may be made from cobalt chromium, nitinol, or stainless steel, for example, and distal elements 20 may be made from cobalt chromium or stainless steel, for example.

Fixation device 12 may be releasably coupled to shaft 11 such that it can be detached and left behind as an implant to hold the leaflets LF together in the coapted position. In this regard, fixation device 12 may be delivered to a target valve percutaneously using any one of a number of different approaches, such as via a transfemoral, a transapical, or a transjugular approach, for example. Thus, in one example of treating MVR, fixation device 12 may be delivered to the deficient mitral valve MV using a transfemoral approach in which fixation device 12 is guided through the inferior vena cava IVC (see FIG. 1A), across the interatrial septum S, and into left atrium LA where fixation device 12 is advanced into the mitral valve MV. Also, in one example of treating TVR, fixation device 12 may be guided transfemorally through the inferior vena cava IVC to the right atrium RA where fixation device 12 is advanced to a desired position within the tricuspid valve TV.

FIG. 3B is an atrial-side view of fixation device 12 in one example of a desired orientation in relation to adjacent leaflets LF of an atrioventricular valve, such as the depicted mitral valve MV. The distal and proximal elements 20, 40 are positioned to be substantially perpendicular to the line of coaptation LOC. Thus, in the case of a mitral valve MV, fixation device 12 may be oriented perpendicular (+/−5 degrees) to a line of coaptation LOC between the posterior leaflet PL and anterior leaflet AL, and in the case of a tricuspid valve TV, fixation device 12 may be positioned perpendicular (+/−5 degrees) to a line of coaptation between the septal leaflet SL and the anterior leaflet AL, the septal leaflet SL and the posterior leaflet PL, or the anterior leaflet AL and the posterior leaflet PL, for example. Device 12 may be moved roughly along the line of coaptation LOC to the location of regurgitation. The leaflets LF may be held in place so that, during diastole, the leaflets LF remain in position between elements 20, 40 surrounded by openings O (also referred to herein as orifices) which result from the diastolic pressure gradient. Advantageously, leaflets LF are coapted such that their proximal or upstream surfaces face each other in a vertical orientation, parallel to the direction of blood flow through the valve. The upstream surfaces may be brought together so as to be in contact with one another or may be held slightly apart but will preferably be maintained in the vertical orientation in which the upstream surfaces face each other at the point of coaptation. This simulates the double orifice geometry of a standard surgical bow-tie repair. Color Doppler echo will show if the regurgitation of the valve has been reduced. If the resulting flow pattern is satisfactory, the leaflets LF may be fixed together in this orientation. If the resulting color Doppler image shows insufficient improvement in valve regurgitation, fixation device 112 may be repositioned. This may be repeated until an optimal result is produced wherein the leaflets LF are held in place.

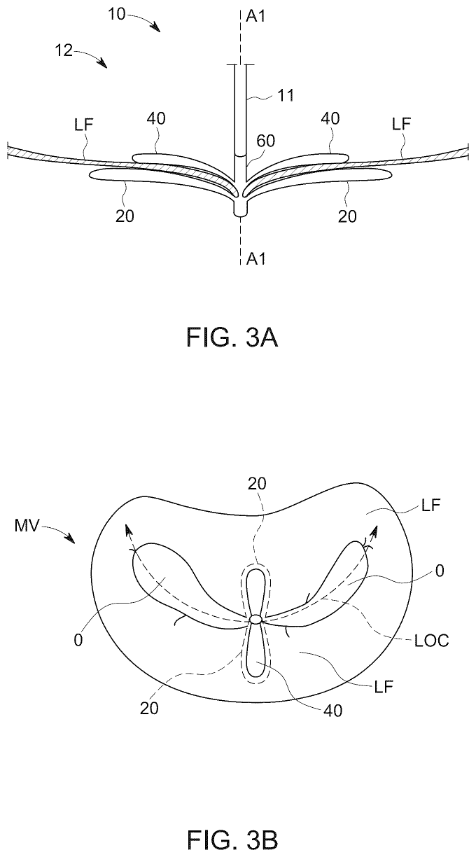

FIGS. 4A-19C depict a fixation device 112 according to another embodiment of the present disclosure. Fixation device 112 may generally include a pair of distal elements 120, a pair of proximal elements 140, a coupling member 160, an actuator 113, and a stud 131. Distal elements 120 may include elongate arms 121 in which each arm has a proximal end portion 121a, which may be rotatably connected to the coupling member 160, and a free end 121b, as best shown in FIG. 4A. Free ends 121b may each have a rounded shape to minimize interference with and trauma to surrounding tissue structures according to one example. In one example, each free end 121b defines a curvature extending about two axes 126, 127. The first axis 126 may be a longitudinal axis of each respective arm 121. Additionally, arms 121 may each include an engagement surface 125 that may also be curved about first axis 126 and may extend at least partially along a length of arm 121 to the free end 121b. Thus, in some examples, engagement surfaces 125 may each have a cupped or concave shape which may maximize contact area engagement with tissue and may assist in grasping and holding valve leaflets. Such cupped or concave shape may further allow arms 121 to nest around shaft 111 of interventional tool 110 while in the closed position to minimize the profile of device 112. Thus, arms 121 may be at least partially cupped or curved inwardly about their longitudinal axes 126 which may form a concavity extending along axis 126 which may nest proximal elements 140 when in a lowered position thereof. The second axis 127 about which each free end 121b may be curved may extend perpendicular to first axis 126, as is also shown in FIG. 4A. The curvature about this second axis 127 may be a reverse curvature located at the most distal portion of free ends 121b. In addition to the dual curvature, free ends 121b may flare outwardly at their respective longitudinal edges. It is believed that both the reverse curvature and flare help create an atraumatic configuration that minimizes trauma to the tissue engaged therewith.

In the nonlimiting embodiment depicted, a transverse width across engagement surfaces 125 (which is in the direction of second axis 127 and determines the width of tissue engaged) may be at least about 2 mm, 3-10 mm in some examples, and about 4-6 mm in some examples. In some embodiments, a wider engagement may be desired wherein the engagement surfaces 125 are larger, for example about 2 cm, or multiple fixation devices 112 may be used adjacent to each other. Arms 121 may also have a length of about 6-12 mm (defined along first axis 126), and engagement surfaces 125 may be configured to engage a length of tissue of about 4-10 mm along the longitudinal axis 126 of arms 121 according to some examples. Also, as shown in the illustrated example, each arm 121 may include a plurality of openings 128 to enhance grip and to promote tissue ingrowth following implantation.

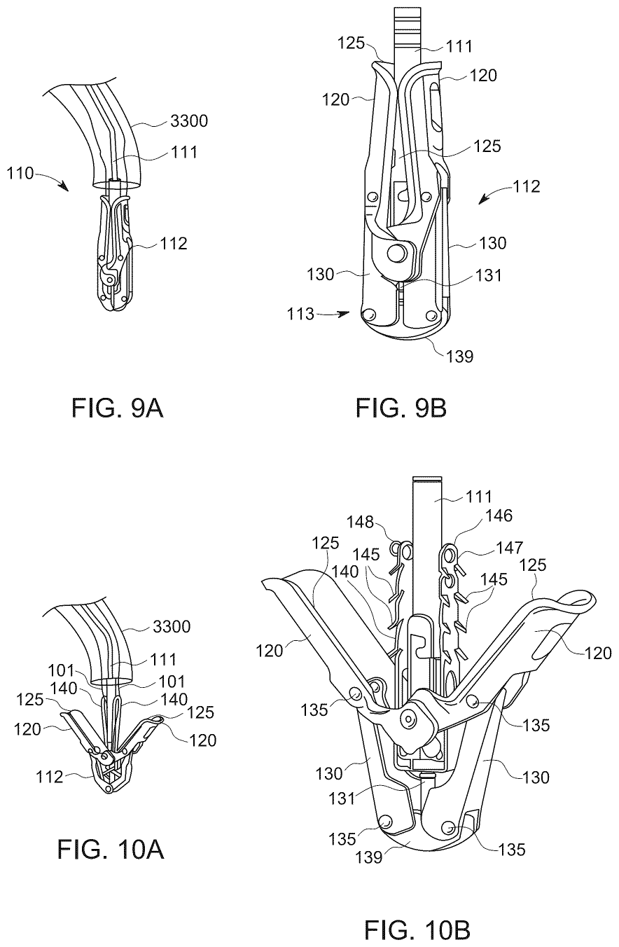

In one example, actuator 113 may include two link members or legs 130. Legs 130 may be comprised of a rigid or semi-rigid metal or polymer such as Elgiloy®, cobalt chromium or stainless steel, however any suitable material may be used. Each leg 130 may have a first end 132, which may be rotatably joined with one of the distal elements 120 at a riveted joint 135, and a second end 134, which may be rotatably joined with stud 131, as shown in FIG. 4A. Although the depicted embodiment shows both legs 130 pinned to stud 131 by a single rivet 135, it is also contemplated that each leg 130 may be individually attached to the stud 131 by a separate rivet, pin or the like. In other embodiments of actuator 113, actuator 113 may include a base 139, and second ends 134 of legs 130 may be rotatably joined with base 169, such as by one or more riveted joints 135, as best shown in FIG. 10B. An actuator rod 170 of delivery 600 may be joinable with actuator 113 directly, such as via direct connection with base 139, or indirectly, such as via connection with stud 131, which itself may extend from base 139. In either of these embodiments, actuator rod 170 may be axially extendable and retractable in a proximal-distal direction to actuate actuator 113 and consequently rotate distal elements 120 between open, closed, and inverted positions, which are described further below. Additionally, coupling member 160, stud 131, and/or base 169 may comprise a center portion or center body of fixation device, for example.

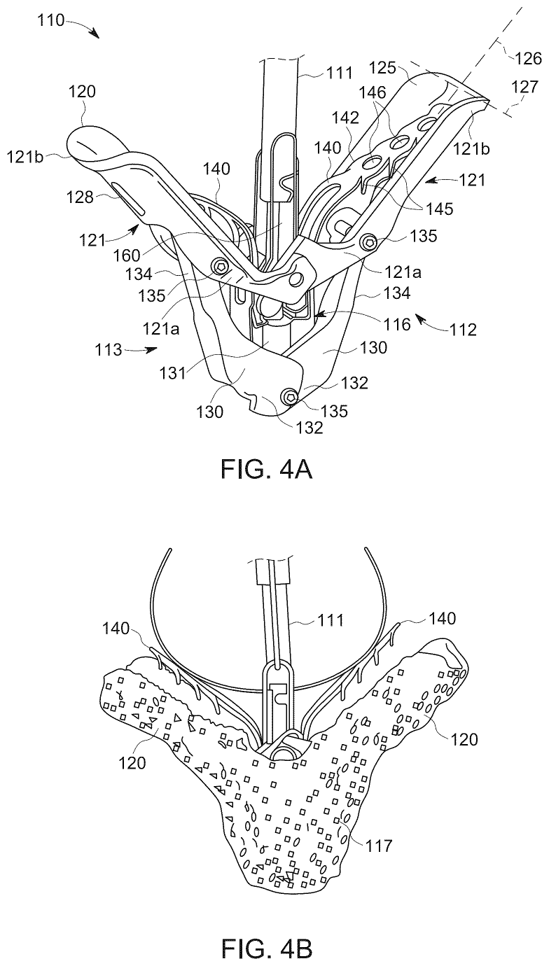

Proximal elements 140 may, in some examples, be flexible, resilient, and cantilevered from a center of fixation device 112. For example, FIGS. 5A and 5B depict a gripping device 114 according to an embodiment of the present disclosure that may generally include a pair of proximal elements 140, a base section 150, and a pair of arm bend features 153 partitioning proximal elements 140 from base section 150.

Proximal elements 140 may be in the form of elongate arms 141 that each extend along a longitudinal axis A2 from a first end portion or fixed end 141a to a second end portion or free end 141b, as shown in FIG. 5A. Each proximal element 140 may also have opposed side edges 142 that define a width transverse to the longitudinal axis A2. Such width may be less than the width of a corresponding distal element 120 such that proximal element 140 may be recessed within the concavity formed by engagement surface 125 of distal element 120 when proximal element 140 is moved into a lowered position, as described in more detail below.

Proximal elements 140 may also each have a first side or proximal side 143 and a second side or distal side 144. In one example, proximal elements 140 may include a plurality of openings 146 that may extend from proximal side 143 to distal side 144, as shown in FIG. 5A. Such openings 146 may be used to couple a proximal element line, which is discussed further below, to a proximal element 140 for raising and lowering proximal element 140. Each proximal element 140 may also include one or more frictional elements 145 extending from distal side 144. For example, each proximal element 140 may include one or more rows of frictional elements 145 where frictional elements 145 in each row may be aligned in a direction transverse to longitudinal axis A2. Frictional elements 145 in such rows may also be aligned with frictional elements 145 in other rows in a lengthwise direction thereby forming columns of frictional elements 145. For example, in the embodiment depicted in FIGS. 5A and 5B, each proximal element 145 may include four rows of two frictional elements 145. In other words, two columns of four frictional elements 145. In other embodiments, proximal elements 140 may include one to six rows of two to six frictional elements 145 per row, for example. However, in other embodiments, frictional elements 145 may be arranged in an offset relationship in a lengthwise and/or transverse direction such that at least some frictional elements 145 are not aligned with another frictional element 145 in such directions.

Frictional elements 145 may comprise frictional protrusions or tines having tapering pointed tips extending from distal side 144 of proximal elements 140. Frictional elements 145 may also be angled toward fixed end 141a of proximal element 140 which may help prevent frictional elements 145 from inadvertently snaring tissue during repositioning of fixation device 112. In one example, frictional elements 145 may be integral with or connected to a distal surface 144 of a proximal element 140 and protrude therefrom. In another example, as shown in FIG. 5A, frictional elements 145 may be formed from side edges 142, such as by cutting and bending the base material forming proximal elements 140, for example. It may be appreciated that any suitable frictional elements may be used, such as prongs, windings, bands, barbs, grooves, channels, bumps, surface roughening, sintering, high-friction pads, coverings, coatings, or a combination of these. However, it should be noted that some types of frictional elements that can be utilized may permanently alter or cause some trauma to the tissue engaged. Thus, it is preferable that frictional elements 145 be atraumatic and generally frictional rather than penetrative so as to not injure or otherwise affect the tissue in a clinically significant way.

Base section 150 may be connected to a center portion or center body of fixation device 112 such that proximal elements 140 extend outwardly therefrom. For example, base section 150 may be coupled to coupling member 160. In the embodiment depicted, base section 150 may include a first member 152, a second member 154, and a third member 156. First and third members 152, 156 may be connected to second member 154 to form a generally U-shaped or box-shaped structure which may allow a lock (discussed below) to be positioned between first and third members 152, 156. However, other shapes may be formed, such as a V-shape, a crescent shape, or semicircular, for example. In some embodiments, first and third members 152, 156 may be connected to second member 154 via base bend features 157, for example. Also, second member 154 may include an opening 158 extending therethrough for receipt of stud 131 and/or actuator rod 170, as shown in FIG. 5A.

Arm bend features 153 may couple a respective proximal element 140 and base section 150. For example, an arm bend feature 153 can couple a proximal element 140 to first member 152 of base section 150, and another arm bend feature 153 can coupled the other proximal element 140 to third member 156 of base section 150. As shown, arm bend features 153 may form a living hinge about which proximal elements 140 may bend relative to base section 150. In this regard, arm bend features 153 may be integral with proximal elements 140 and base section 150 and may bias proximal elements 140 to a relaxed position. As illustrated in FIG. 5B, proximal elements 140 may form a relaxed angle 149 formed between proximal sides 143 of each proximal element 140. Such relaxed angle 149 is formed when proximal elements 140 are in the relaxed position and may form an angle of about 85 degrees to 200 degrees (+/−5 degrees). For example, proximal elements 140 may form a relaxed angle of 180 degrees in the relaxed position. In another example, proximal elements 140 may form a relaxed angle of 185 degrees in the relaxed position. Although the embodiment depicted illustrates bend features 153 as living hinges, in other embodiments bend features 153 may comprise a biased hinge that modularly connects proximal elements 140 to base section 150. For example, proximal elements 140 may be separately formed from base section 150 and modularly connected to base section 150 via arm bend features 153 which may each comprise a spring biased hinge biasing a respective proximal element 140 to the relaxed position, for example.

Arm bend features 153 may also each include an elongate opening extending 151 along the longitudinal axis A2 which may furcate each arm bend feature 153, as illustrated in FIG. 5A. Such an elongated opening 151 may have a uniform width extending along axis A2. However, in some embodiments, such as the embodiment depicted, elongate opening 151 may form a bowling-pin shape such that a width of opening 151 is narrower at one end (e.g., the end closest to free end 141b) than the other end (e.g., the end furthers from free end 141b) and is wider somewhere in between. Elongate opening 151 may also not be relegated to just arm bend feature 153 but may also extend from arm bend feature 153 to proximal element 140 and/or base body 150. The elongate opening 151 and corresponding furcation of arm bend features 153 may be configured (e.g., in size, shape, spacing, position, etc.) so as to provide the desired resiliency, fatigue resistance, and/or flexibility at the coinciding arm bend features 153.

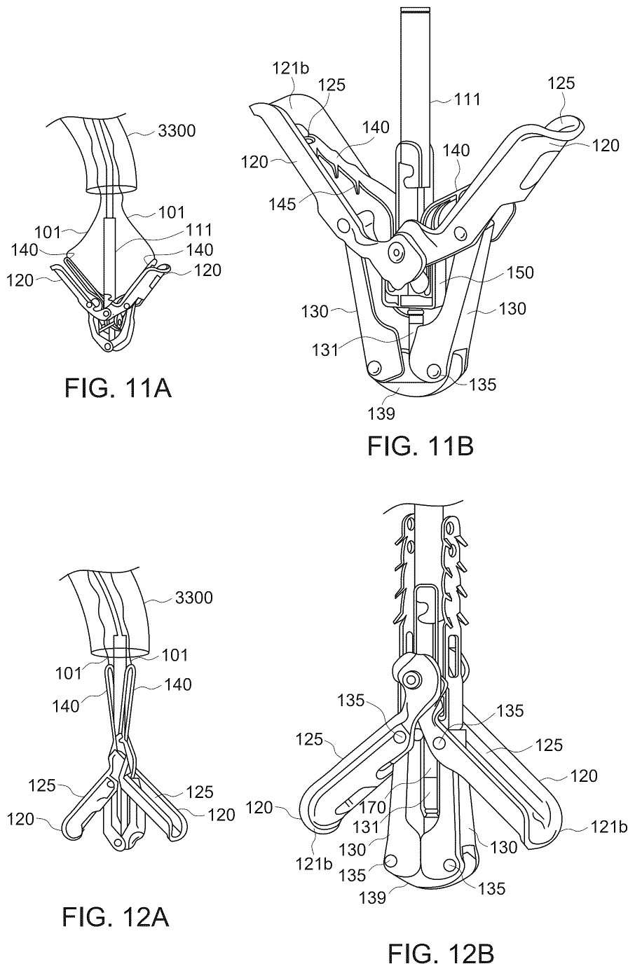

Base bend features 110 and arm bend features 112 may be configured to give gripping device 116 a bent configuration when gripping device is in a relaxed state (i.e., when proximal elements are in the relaxed position), such that when gripping device 114 is forced into a stressed state (e.g., by bending proximal elements at one or more of the base and/or arm In the exemplary embodiment depicted, gripping device 114 may be formed from a metallic sheet of a spring-like material, such as a shape-memory metal (e.g., Nitinol) which may provide the bias of proximal elements 140 toward the relaxed position. Alternatively, gripping device 114 could be molded from a biocompatible polymer. Each proximal element 112 may, in one example, be configured to be at least partially recessed within the concavity of the distal element 120 when no tissue is present. When fixation device 112 is in the open position, each proximal element 140 may be separated from the engagement surface 125 near free end 121b of arm 121 and may slope toward engagement surface 125 near free end 121b with the free end 141b of proximal element 140 contacting engagement surface 125, as illustrated in FIGS. 4A and 11B. This arrangement may be facilitated by the dimensions of base section 150. For example, increasing or decreasing the respective lengths of first, second, and third members 152, 154, 156 of base section 150 may increase or decrease the separation distance between a proximal element 140 and corresponding distal element 120 which may help accommodate a valve leaflet or other tissues of varying thicknesses. Further examples of gripping devices that may be utilized in fixation device 112 are described in more detail in U.S. Pat. No. 11,096,691, the disclosure of which is incorporated by reference herein in its entirety.

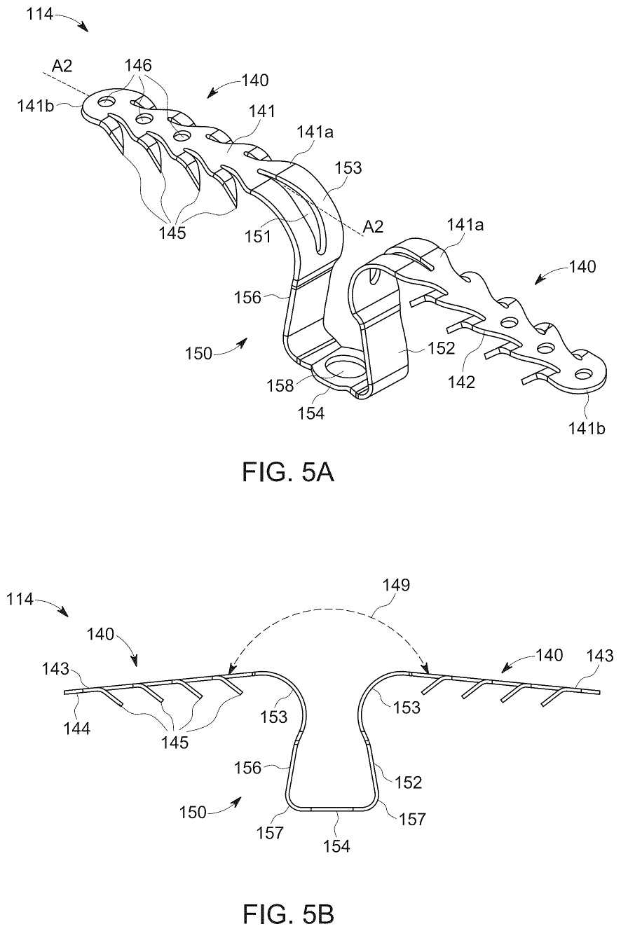

In other embodiments proximal elements may be connected to or otherwise extend from distal elements rather than from a center of fixation device, like that of fixation device 112. For example, FIGS. 6A and 6B depict a gripping device 214 according to another embodiment of the present disclosure that may generally include a first arm 240, a second arm 250, and an arm bend feature 260 partitioning first arm 240 from second arm 250. Gripping device 214 may be made from a shape-memory-metal material, such as Nitinol, for example.

First arm 240 may constitute a proximal element of fixation device 112, like that of and as an alternative to proximal element 140 and may include one or more frictional elements 245 which may be similar to frictional elements 145 discussed above. Thus, a plurality of frictional elements 245 may extend from a distal side of first arm 240 such as in one or more rows and/or columns. In the embodiment depicted, a single row of three frictional elements 245 may be provided near a free end 241b of first arm 240. But, as mentioned above, first arm 240 may have any number of frictional elements 245, such as two, four, or six, for example. First arm 240 may also include a pair of elongate members 247 offset from each other to form a space 248 therebetween. Such space 248 may be configured to receive second arm 250, for example. Additionally, first arm 240 may include one or more openings 246, such as near free end 241b, as shown in FIG. 6A. Such opening 246 may be configured to receive a proximal element line for raising and lowering first arm 240.

Second arm 250 may be in the form of a beam or other elongate structure. Second arm 250 (also referred to herein as base section) may be configured to couple to a distal element 120. For example, in the embodiment depicted in FIGS. 6A and 6B, second arm 250 may be curved in a plane transverse to its longitudinal axis. For example, second arm 250 may be semi-cylindrical such that it may have a semi-circular profile. Thus, second arm 250 may have a convex surface 255 configured to conform to the cupped curvature of engagement surface 125 of a corresponding distal element 120. FIG. 6B illustrates second arm 250 coupled to proximal engagement surface 125 of distal element 120 such that it is generally recessed within distal element 120 and free ends 241b, 251b of first and second arms 240, 250 point in the general direction toward free end 121b of distal element 120. Thus, in some embodiments, second arm 250 may have a width configured to be positioned within the concavity of distal element 120 and secure to proximal engagement surface 125. In other embodiments, a second arm 250′ of an alternative gripping device 214′ may not be concave and may instead have a planar surface corresponding to a planar engagement surface 125′ of an alternative distal element 120′ and secured thereto, as illustrated in FIG. 6C. In further embodiments, distal element 120 may include a recess or pocket for receipt and securement of second arm 250, such as in a press-fit manner, for example. Second arm 250 may be secured to distal element 120 in any number of ways, such as via one or more sutures, welding, press-fit, fastener (e.g., rivet or screw) or the like. For example, a rivet, screw, or suture may pass through one or more openings 257 in second arm 250 and into distal element 120. A tissue fixation device, such tissue fixation device 112, may include a pair of gripping devices 214 with one coupled to each distal element 120 as mentioned above.

Arm bend feature 260 may be coupled to a fixed end 241 of first arm 240 and a fixed end 251a of second arm 250 such that first and second arms 240, 250 extend in the same general direction and may form a V-shape when first arm 240 is in an exemplary open or raised position, as illustrated in FIGS. 6B and 6C. As shown, arm bend feature 260 may form a living hinge about which first arm 240 may bend relative to second arm 250. In this regard, arm bend feature 260 may be integral with first arm 240 and second arm 250 so as to form a monolithic structure and may bias first arm 240 to a relaxed position. Such relaxed position may include second arm 250 extending through space 248 between elongate members 247 of first arm 240 to form an X-shape. However, it should be noted that such position can generally only be achieved when gripping device 214 is not coupled to distal element 120 as the presence of distal element 120 would prevent second arm 250 from passing into space 248. It should also be appreciated that in some embodiments of gripping device 214, arm bend feature 260 may be a spring loaded or otherwise biased hinge coupling separately formed first and second arms 240, 250.

Fixation device 114 may also have a covering 117, as shown in FIG. 4B. As depicted, covering 117 may encapsulate distal elements 120 and actuator 113. Thus, engagement surfaces 125 may be covered by covering 117 which may help minimize trauma on tissues and enhance primary fixation via additional friction to assist in grasping. Additionally, covering 117 on engagement surfaces 125 may facilitate tissue ingrowth to provide for secondary fixation to ensure long-term security. Covering 117 may be loosely fitted and/or may be flexible such that device 112 can freely move to various positions all the while covering 117 conforms to the contours of the device 112 and remains securely attached thereto. It may be appreciated that the covering 117 may cover specific parts of fixation device 112 while leaving other parts exposed. For example, proximal elements 140 may be exposed, while distal elements 120 and actuator 113 may be covered. However, in some embodiments, proximal elements 140 may be covered with covering 117 to enhance grip and tissue ingrowth following implantation. Preferably, when a covering 117 is used in combination with frictional elements 145 or other frictional features, such as those extending from proximal elements 140, such features may protrude through such covering 117 so as to contact any tissue engaged by proximal elements 140.

Covering 117 may be comprised of any biocompatible material, such as polyethylene terepthalate, polyester, cotton, polyurethane, expanded polytetrafluoroethylene (ePTFE), silicon, or various polymers or fibers and have any suitable form, such as a fabric (woven or unwoven), mesh, textured weave, felt, looped or porous structure. Generally, covering 117 has a low profile so as not to interfere with delivery through an introducer sheath or with grasping and coapting of leaflets or tissue. Covering 117 may alternatively be comprised of a polymer or other suitable materials dipped, sprayed, coated, or otherwise adhered to the surfaces of the fixation device 112. Optionally, a polymer coating may include pores or contours to assist in grasping the tissue and/or to promote tissue ingrowth. Any of the coverings 117 may optionally include drugs, antibiotics, anti-thrombosis agents, or anti-platelet agents such as heparin, COUMADIN® (Warfarin Sodium), to name a few. These agents may, for example, be impregnated in or coated on the coverings 117. These agents may then be delivered to the grasped tissues surrounding tissues and/or bloodstream for therapeutic effects.

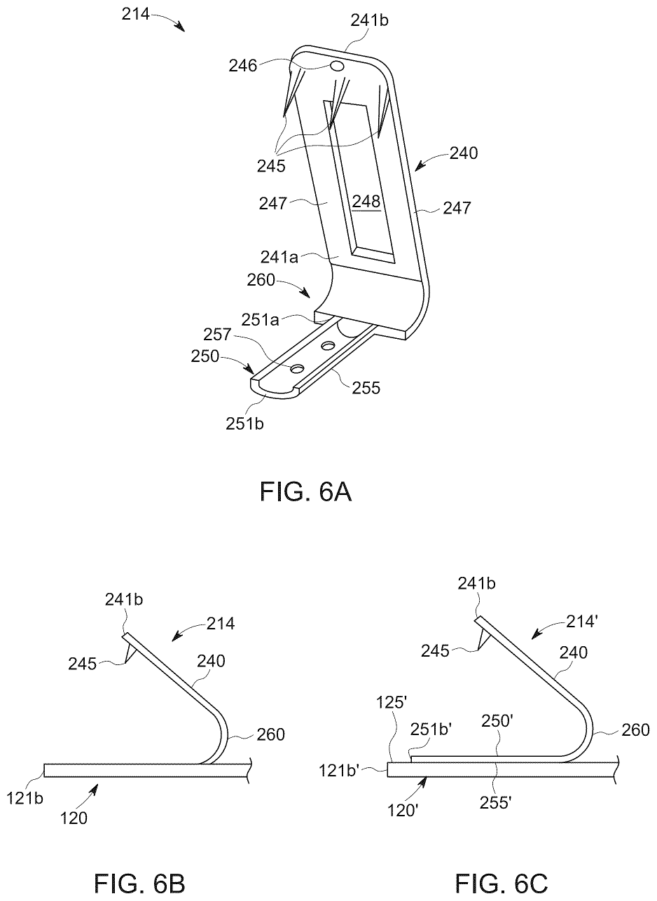

FIGS. 7A-7C depict an exemplary coupling system 115 between fixation device 112 and delivery system shaft 111. As mentioned above, once the leaflets of a target valve are coapted in the desired arrangement, fixation device 112 may then be detached from delivery 600 and left behind as an implant to hold the leaflets together in the coapted position. Such detachment may occur between coupling member 160 of fixation device 112 and a distal end of delivery shaft 111. Thus, coupling member 160 may be configured to be releasably coupled to shaft 111. Coupling member 160 may be disposed at a center of fixation device 112 and may extend proximally along it's the longitudinal axis of fixation device 112. In the coupling system 115 depicted, shaft 111 may form a tubular upper shaft with a first mating surface 163 formed at a distal end thereof, and coupling member 160 may form a detachable lower tubular shaft with a second mating surface 162 formed at a proximal end thereof. Mating surfaces 162, 163 may be correspondingly shaped so that they interlock and form a joining line 165 when merged together, as shown in FIG. 7B. In this regard, mating surfaces 162, 163 may have any shape or curvature which allows or facilitates interlocking and later detachment. For example, in the depicted embodiment, mating surfaces 162, 163 define a joining line 165 with an S-shaped curvature.

Coupling system 115 may also include actuator rod 170 and stud 131 (or alternatively base 139) such that fixation device 112 may also be releasably coupled to delivery 600 via connection between actuator rod 170 and stud 131. When shaft 111 is coupled to coupling member 160, they may collectively form an axial channel. Actuator rod 170 may pass through this channel to bridge the joining line 165, as shown in FIG. 7B. Actuator rod 170 may comprise a proximal extremity 171, a distal extremity 172, and a joiner 174. Distal extremity 172 may be smaller in diameter than proximal extremity 171 and may be optionally surrounded by a coil 173 which may serve to bias joiner 174 in a proximal direction. However, in some embodiments, actuator rod 170 may not have coil 173 or proximal and distal extremities 171, 172 of differing diameters. Joiner 174 may be removably coupled with stud 131 of fixation device 112 via any one of various possible release mechanisms. For example, in the embodiment depicted, joiner 174 may be threadedly connected to stud 131 of fixation device 112. In this regard, joiner 174 may have internal threads 175 which mate with external threads 133 on stud 131. Alternatively, joiner 174 may have external threads which mate with internal threads of stud 131. As described previously, stud 131 may be connected with distal elements 120 so that advancement and retraction of stud 131, by means of actuator rod 170, manipulates distal elements 120. It is also contemplated that joiner 174 may be directly threadedly engaged with base 139 where no stud 131 is provided. Once detachment of fixation device 112 is desired, actuator rod 170 may be rotated until threads 175 of joiner 174 disengage threads 133 of stud 131. Actuator rod 170 may then be retracted to a position above mating surfaces 162, 163 which in turn allows coupling member 160 to separate from shaft 111 along joining line 165, as illustrated in FIG. 7C.

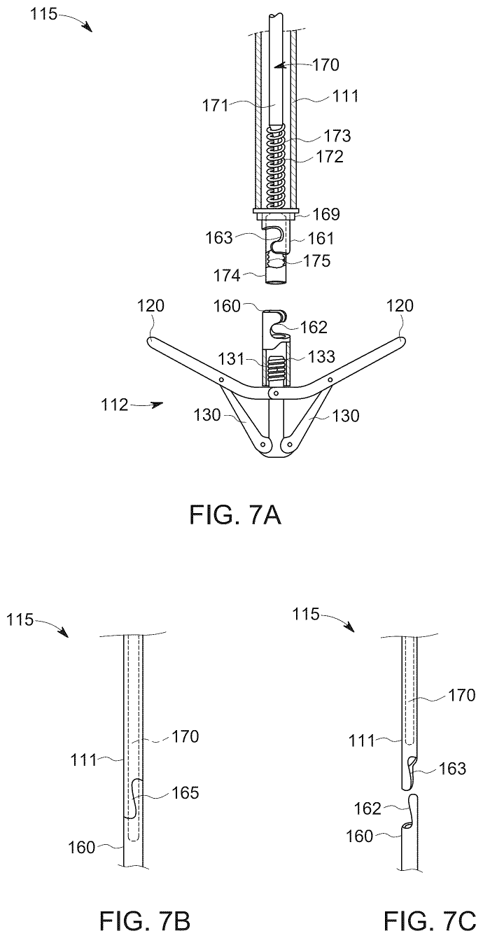

FIGS. 8A and 8B illustrate an alternative example of a coupling system. In this exemplary coupling system 315, shaft 311 of the delivery system (e.g., delivery 600) may be releasably coupled with coupling member 360 via a detent mechanism, for example. In this regard, shaft 311 may form an upper tubular shaft with detent mechanism features and coupling member 360 may form a lower tubular shaft with detent mechanism features configured to releasably connect with the detent mechanism features of shaft 311. In the embodiment depicted, the detent mechanism may include one or more spring arms 361 integrally formed on shaft 311 and one or more receptacles 362 sized to receive spring arms 361 within coupling member 360. However, shaft 311 may include receptacles 362, while coupling member 360 may include spring arms 361, for example. As shown, spring arms 361 may have a flange-like engagement element 363 at a distal end thereof and are preferably biased inwardly, i.e., toward an interior shaft 311, as shown in FIG. 8B. Receptacles or apertures 362 may be configured to receive and mate with respective engagement elements 363 of spring arms 361, as shown in FIG. 8A. Receptacles 362 may extend all the way through the wall of coupling member 360 and may be sized to snuggly fit both engagement elements 362. A snuggly fitting rod (such as actuator rod 370) may extend through shaft 311 and coupling member 360 and may outwardly deflecting the inwardly biased spring arm(s) 361 such that the engagement elements 363 are pushed into respective engagement with a corresponding receptacle 362 thereby coupling the shaft 311 to coupling member 360, as shown in the example of FIG. 8A. When desirable to detach fixation device 112 from delivery 600, actuator rod 370 may be retracted to a position above spring arm(s) 361 and engagement features 363 thereof. This allows the inwardly biased spring arms 361 and corresponding engagement elements 363 to disengage from receptacles 362 thereby detaching shaft 311 and coupling member 360. As mentioned above, actuator rod 370 may be threadedly engaged to stud 131. Thus, actuator rod 370 may first be rotated to unthread its threads 375 from stud 131 and then retracted to release coupling member 360 according to an example of the disclosure.

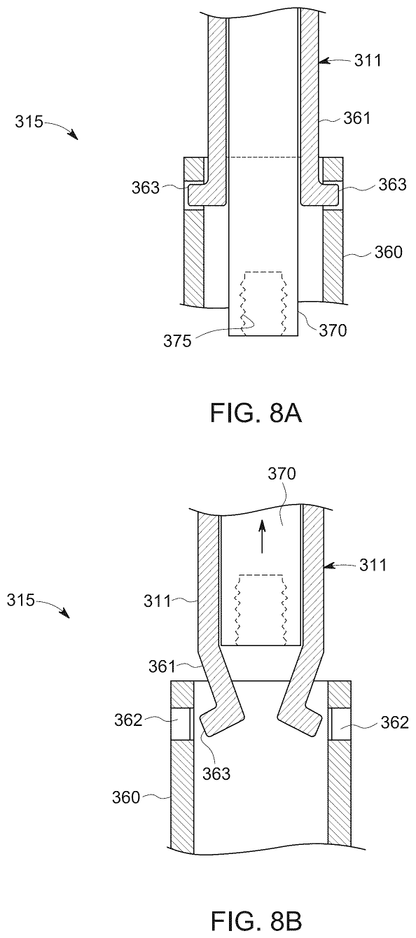

As mentioned above, fixation device 112 may, in one example, be actuated through multiple positions within a mammalian body during a transcatheter procedure such as by extending and retracting actuator rod 170 when coupled to stud 131 and/or base 139. FIGS. 9A-9B, 10A-10B, 11A-11B, 12A-12B, and FIGS. 13A-13B illustrate several of these possible positions and in a sequence that may be utilized during a transcatheter, therapeutic procedure (e.g., tissue approximation).

FIGS. 9A and 9B depict fixation device 112 in an example of a closed position or delivery position. Fixation device 112 may assume the closed position when being delivered through a guide catheter or sheath 3300 of a steerable guide system. In the closed position, the opposed pair of distal elements 120 may be positioned so that engagement surfaces 125 thereof face each other. The cupped or concave shape of each arm 121 in this example allows arms 121 to surround shaft 111 and optionally contact each other on opposite sides of shaft 111. This provides a low profile for fixation device 112 so that it is readily passable through a delivery catheter 3300 and through any anatomical structures, such as those within the cardiovascular system.

FIGS. 10A-10B depict fixation device 112 in an example of an open position. Fixation device 112 may assume the open position for capturing and grasping leaflets of a heart valve. In an open position, distal elements 120 may be rotated so that engagement surfaces 125 thereof face a first direction such that engagement surfaces 125 are disposed at an acute angle relative to shaft 111. For example, the acute angle formed between each engagement surface 125 and shaft may be 45 degrees to 90 degrees. Stated differently, in the open position, engagement surfaces 125 of distal elements 120 may be oriented 90 degrees to 180 degrees relative to each other. However, it is generally preferable for arms to be positioned 120 degrees relative to each other (and 60 degrees relative to shaft 111) for capturing leaflets. Movement of fixation device 112 from the closed position to the open position may be achieved by advancing stud 131 distally relative to coupling member 160 by distally advancing actuator rod 170. Conversely, fixation device 112 may be moved from the open position to the closed position by retracting actuator rod 170 and retracting stud 131 proximally, according to one example of the disclosure.

As shown in FIG. 10B, proximal elements 140 (or proximal elements 240) may be in a raised or insertion position when fixation device 112 is in the open position to facilitate insertion of leaflets between distal and proximal elements 120, 140 for their capture. A loop 148 may be provided on one or both proximal elements 140 for receipt of a proximal element line that can raise and lower proximal elements 140. Proximal elements 140 are, in one example, biased toward distal elements 120. In this regard, proximal elements 140 may be moved inwardly toward shaft 111 and held against shaft 111 with the aid of proximal element lines 101 which can be in the form of sutures, wires, nitinol wire, rods, cables, polymeric lines, or other suitable structures, as shown in FIG. 10A. Thus, FIGS. 10A and 10B depict fixation device 112 in an insertion configuration in which proximal elements 140 are in a raised position and distal elements 120 are in an open position.

Once fixation device 112 has been positioned in a desired location against the valve leaflets, the leaflets may then be captured between proximal elements 140 and distal elements 120. FIGS. 11A and 11B illustrate fixation device 112 in an example of such a position. Here, proximal elements 140 are lowered toward engagement surfaces 125 so that proximal elements 140 are in a lowered or capture position, and the leaflets are held between distal and proximal elements 120, 140. Proximal elements 140 are, in one example, lowered into the lowered position while distal elements 120 remain in the open position. Thus, fixation device 112, as shown in FIGS. 11A and 11B is in an example of a capture configuration which may be similar to the insertion configuration of FIGS. 10A and 10B, but with the difference being that proximal elements 140 are now lowered toward distal elements 120 by releasing tension on proximal element lines 101 to compress the leaflet tissue therebetween. At any time, the proximal elements 140 may be raised and the distal elements 120 adjusted or inverted to reposition fixation device 112 if regurgitation is not sufficiently reduced according to one example of the disclosure.

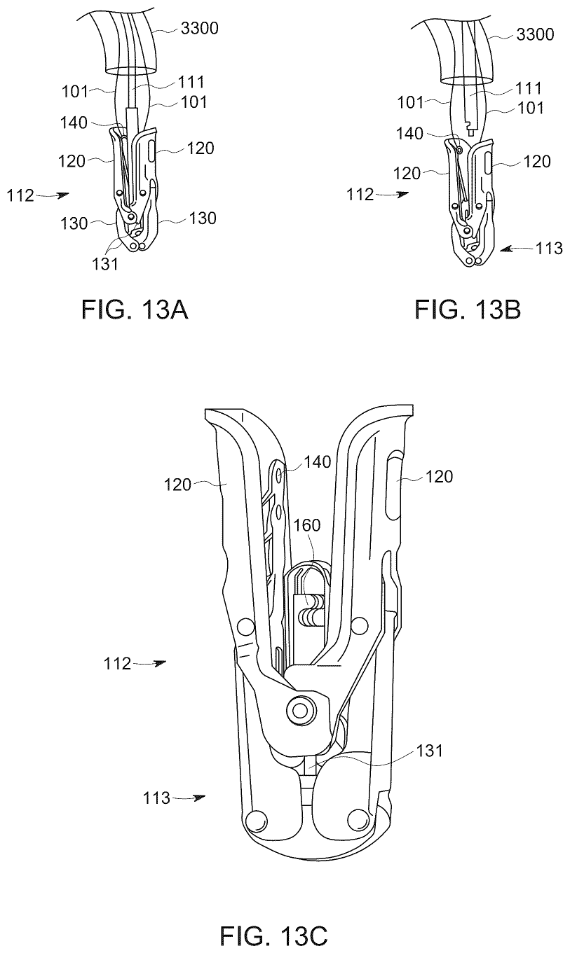

FIGS. 12A-12B depict an example of an inverted position of fixation device 112. Fixation device 112 may assume the inverted position to aid in repositioning or removal of fixation device 112. In one example of the inverted position, distal elements 120 may be further rotated from the open position, which may be achieved by advancing stud 131 further relative to the open position, so that the engagement surfaces 125 of distal elements 120 face outwardly, and free ends 121b point distally. Additionally, in some examples, engagement surfaces 125 of each arm 121 may form an obtuse angle relative to shaft 111. For example, the obtuse angle formed between each engagement surface 125 and shaft 111 may be 135 degrees to 180 degrees. Stated differently, in the inverted position, engagement surfaces 125 of distal elements 120 may be oriented 270 degrees to 360 degrees relative to each other.

Also, as shown in FIG. 12B, in one example proximal elements 140 are in their raised position against shaft 111 while distal elements 120 are in the inverted position by exerting tension on the proximal element lines 101. Thus, a relatively large space may be created between proximal and distal elements 140, 18 for repositioning. In addition, the inverted position allows withdrawal of the fixation device 112 through the valve while minimizing trauma to the leaflets. Engagement surfaces 125 provide an atraumatic surface for deflecting tissue as the fixation device is retracted proximally. It should be further noted that tines 145 of proximal elements 140 may, in some examples, be angled slightly in the distal direction (away from the free ends of the proximal elements 140), reducing the risk that tines 145 will catch on or lacerate tissue as fixation device 112 is withdrawn and while proximal elements 140 are in the raised position.

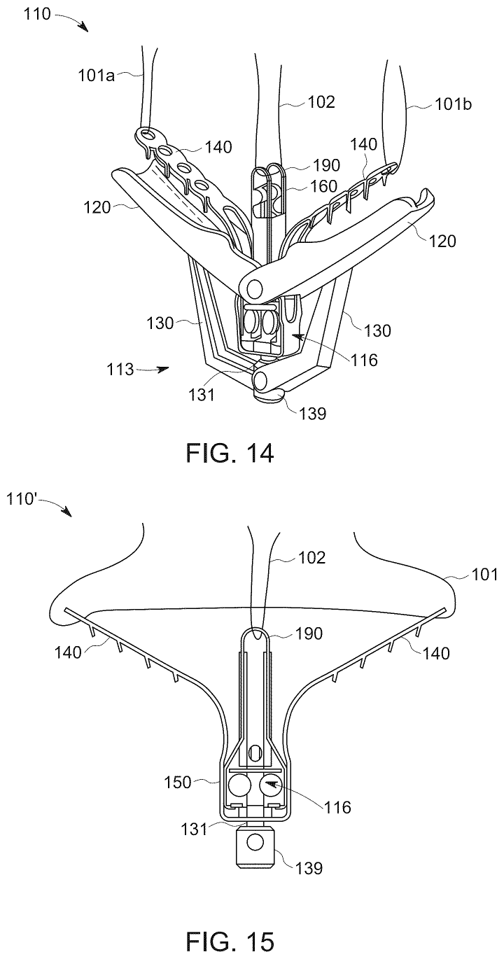

After the leaflets have been captured between distal and proximal elements 120, 140, distal elements 120 may be returned to or toward the closed position where they may be locked in place. An example of such locking is described further below. FIG. 13A illustrates fixation device 112 in the closed position wherein the leaflets (not shown) are captured and coapted. In one example, this is achieved by retraction of the stud 131 proximally relative to coupling member 160 so that the legs 130 of the actuator 113 apply an upwards force to distal elements 120 which in turn rotate distal elements 120 so that engagement surfaces 125 again face one another, similar to that of FIGS. 9A and 9B, and so that distal elements 120 rotate proximal elements 140 in a direction toward shaft 111. However, because the leaflets are captured between distal and proximal elements 120, 140, it may be desirable to keep distal elements 120 at about 20 degrees to 60 degrees relative to each other so as to limit the amount of tension and stress on the native tissue. Thus, while fixation device 112 may be returned to the closed position, such closed position may not be as closed as in the initial delivery position.

As shown in FIG. 13B, fixation device 112 may then be released from shaft 111 of delivery system 600 while in the closed position. As mentioned, fixation device 112 may be releasably coupled to delivery system 600 via a coupling system (e.g., coupling system 115 or 315). When the coupling structures of such coupling system are released, proximal element lines 101 may remain attached to proximal elements 140 following detachment to function as a tether to keep the fixation device 112 connected with the delivery catheter 610 (see FIG. 16) for reconnection and repositioning. However, in other embodiments, proximal elements lines 101 may be released prior to release of fixation device 112 or concurrently with the release of fixation device 112, as described in more detail below.

FIG. 13C illustrates a released fixation device 112 in an example of a closed position. As shown, coupling member 160 remains separated from shaft 111 of delivery system 600, and proximal elements 140 are deployed so that tissue (not shown) may reside between proximal elements 140 and distal elements 120.

As mentioned above, proximal element lines or actuators 101 may be releasably coupled to proximal elements 140. In some examples, proximal element lines 101 may pass through an opening in proximal elements 140, such as openings 146 and 246 in the case of proximal element 240. In other examples, eyelets, which may be formed from one or more lengths of suture, may be coupled to proximal elements 140 and proximal element lines 101 may pass through such eyelets. Thus, proximal element lines 101 may be released from proximal elements 140 prior to, concurrent with, or after release of fixation device 112 from delivery system 600 according to various examples.

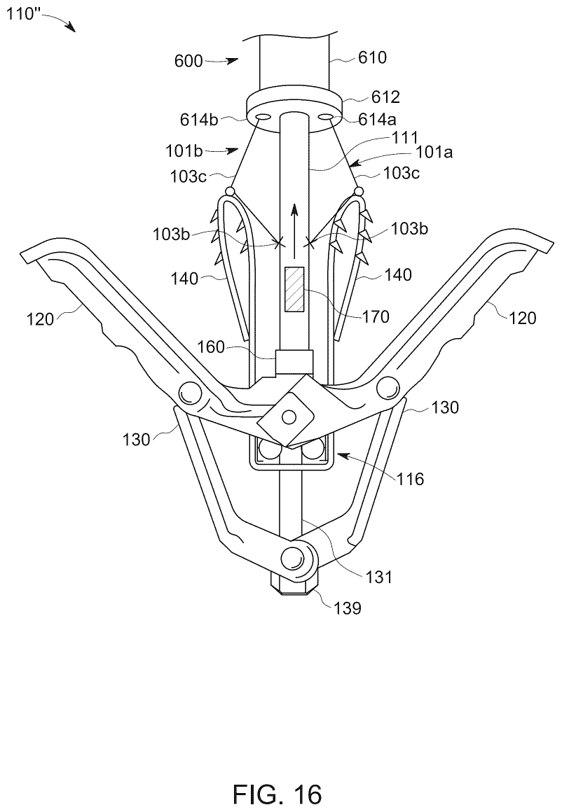

In an exemplary embodiment of interventional tool 110, as shown in FIG. 14, a plurality of proximal element lines 101a, 101b may extend through corresponding lumens 614a, 614b of delivery catheter 610 of delivery system 600 (see FIG. 16) and may be coupled to proximal elements 140 of fixation device 112. Each of proximal element lines 101a and 101b may be elongated flexible threads, wire, cable, sutures, or lines extending through shaft 111, looped through proximal elements 140, and extending back through shaft 111 to a delivery device handle of delivery system 600. When detachment is desired, one end of each proximal element line 101a, 101b may be released from delivery system 600, and the other end pulled to draw the free end distally through shaft 111 and through proximal element 140 thereby releasing it. Also, in this arrangement, proximal element lines 101a and 101b may be independently or concurrently manipulated so as to independently or concurrently raise and lower proximal elements 140, respectively.

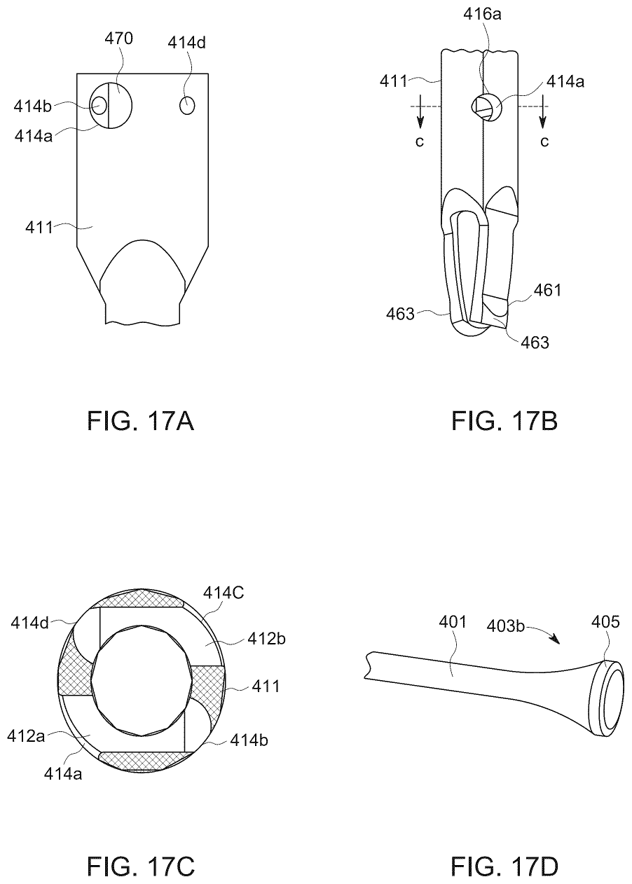

In another example, interventional tool 110′ may be configured, as shown in FIG. 15 with respect to certain components thereof, such that proximal elements 140 may alternatively be supported by a single proximal element line 101 which may extend through both of the proximal elements 140. In this arrangement both proximal elements 140 may be raised and lowered concurrently by action of a single proximal element line 101. Whether proximal elements 140 are manipulated individually by separate proximal element lines 101 or jointly by a single proximal element line 101, the proximal element lines 101 may extend directly through openings (e.g., openings 146, 246) of the proximal elements 140 and/or through a layer or portion of a covering 117 on proximal elements 140, or through a suture loop/eyelet above or below a covering 117, for example.

In a further example, interventional tool 110′ may be configured, as shown in FIG. 16, such that each proximal element line 101a, 101b may be releasably engaged with structures that are activated by removal of the actuator rod 170 that passes through coupling member 160 and shaft 111 such that release of proximal element lines 101a, 101b occurs concurrently with the release of fixation device 112 from delivery system 600. Thus, in one example, which is depicted in FIG. 16, each proximal element line 101a, 101b may have a first end portion 103a (e.g., proximal end portion), which may be coupled to an actuator of a delivery system handle, a second end portion 103b (e.g., distal end portion) which may be releasably engages to shaft 111 via actuator rod 170, and an intermediate portion 103c which may be coupled to a proximal element 140. As described above and as illustrated in FIG. 7A, stud 131 may be releasably attached to actuator rod 170 which passes through coupling member 160 and shaft 111 of delivery system 600. In this way, actuator rod 170 is connectable with fixation device 112 and acts to manipulate fixation device 112 so as to move it through its various positions, which are described above. After the leaflets have been coapted, actuator rod 170 may be removed proximally from stud 131 which may thereby also release coupling member 160 from shaft 111, as described with respect to FIGS. 7A-7C and also FIGS. 8A and 8B with respect to coupling system 315. This action of actuator rod 170 may be utilized to release distal end portion 103b of each of proximal element lines 101a, 101b.

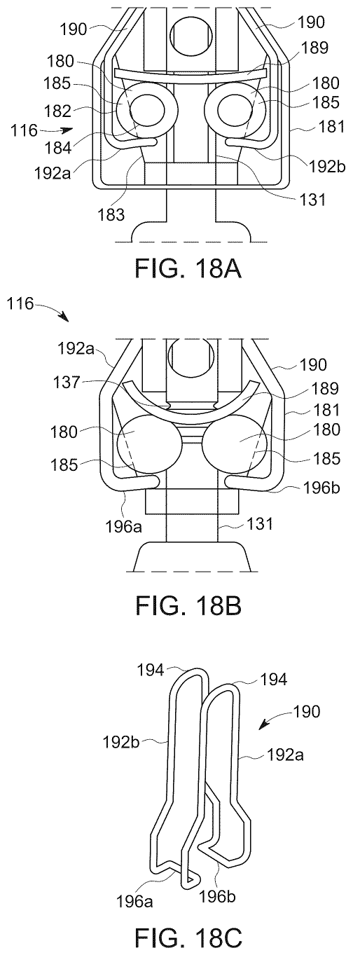

Exemplary features which may be implemented in interventional tool 110′ to facilitate release of proximal element lines 101a, 101b in this manner are shown in FIGS. 17A-17G. As depicted, an actuator rod 470 may be used as an anchor to restrict proximal movement of one or more proximal element lines 401. Proximal element line 401 has a distal end portion 403b which may include a catch element 405, for example a trumpet 405 having a cone shape (see FIG. 17D) or other shapes, such as a ball 405′ having a spherical shape (see FIG. 17G), which can be sized to be received within shaft 411. As shown in the example of FIG. 17B, shaft 411 may have spring arms 461 like that of the coupling system 315 of FIGS. 8A and 8B for releasing device 112 from shaft 411. However, shaft 411 may also have mating surfaces of FIGS. 7A and 7C. In any event, a portion of shaft 411 proximal of spring arms 461 (or mating elements 463), may have two slots 412a and 412b defined therein. Slot 412a can define holes 414a and 414b and slot 412b can define holes 414c and 414d. Holes 414a and 414c can be sized to receive catch element 405 of a pair of proximal element lines 401, respectively, therethrough and into slots 412a and 412b, respectively. Holes 414b and 414d can be sized to prevent catch element 405 of proximal element lines 401, respectively, from extending beyond slots 412a and 412b, respectively. The configuration of slots 412a and 412b and holes 414a-414d can allow for easier manufacture of the features in shaft 411. Slots 412a and 412b can be drilled to ensure that slots 412a and 412b do not pass the entire way through shaft 411. In this example configuration, catch elements 405 of proximal element lines 401 can be maintained within shaft 411 to manage the slack of proximal element lines 401.

In one example, catch element 405 of proximal element line 401 can be inserted into slot 412a through hole 414a beyond a longitudinal axis of shaft 411 and toward hole 414b, and catch element 405 of proximal element line 401 can be inserted into slot 412b through hole 414c beyond the longitudinal axis of shaft 411 and toward hole 414d prior to the insertion and coupling of the actuator rod 470 (which passes through shaft 411) with stud 131 of fixation device 112. With actuator rod 470 extending through shaft 411, actuator rod 470 may directly engage catch elements 405 of lines a plurality of proximal element lines 401 thereby preventing their movement back out along the path through which they were inserted. For example, trumpets 405 can be inhibited from being advanced through holes 414b and 414d, respectively, and can be prevented from being pulled past actuator rod 470 and through holes 414a and 414c, respectively. Accordingly, the second end portions 403b of proximal element lines 401 can be held in place relative to shaft 414. Once the actuator rod 470 is decoupled from stud 131 and subsequently retracted, movement of catch elements 405 at the distal end portions of proximal element lines 401 is no longer restricted and proximal element lines 401 are free to move. Upon proximal retraction, proximal element lines 401 can thread through holes 414a and 414c, respectively, and decouple from the proximal elements 140.

In accordance with one example of the disclosed subject matter, slots 412a and 412b can be drilled at an angle towards the distal end of shaft 411 (see FIGS. 17E and 17F), e.g., with hole 414 b formed distal to hole 414 a on one side, and hole 414 d formed distal to hole 141c on the other side. This example configuration of slots 412a and 412b can provide easier deployment of a plurality of proximal element lines 401 and can reduce friction.

Prior to securing second end portion 403b of each proximal element line 401 with the shaft 411, each proximal element line 401 can be coupled with a respective proximal element 140, such as in the manner described above with respect to FIG. 16. Thus, when proximal element lines 401 are actuated proximally, proximal element lines 401 can move proximal elements 140 relative to distal elements 120, thereby moving proximal elements 140 between their respective raised and lowered positions.

As mentioned above, fixation device 112 optionally includes a lock (e.g., lock 116) for locking device 112 in a particular position, such as in any one of the aforementioned open, closed, and inverted positions or any position therebetween. It may be appreciated that according to various examples, lock 116 may be configured for both locking and unlocking which correspondingly allows device 112 to be both locked and unlocked. As described in more detail below with respect to various lock examples, such locks may have components disposed between coupling member 160 and base 139 which may be configured to selectively arrest proximal-distal movement of stud 131/base 139 which consequently arrests movement of distal elements 120. Such locks may help provide end user control of the final arm angle of fixation device 112 for tailored and optimal results for each patient. Additionally, such locks may bring the leaflets and annulus together which may result in beneficial dimensional changes of the target valve which can prevent adverse remodeling of the heart, particularly for patients with heart failure.

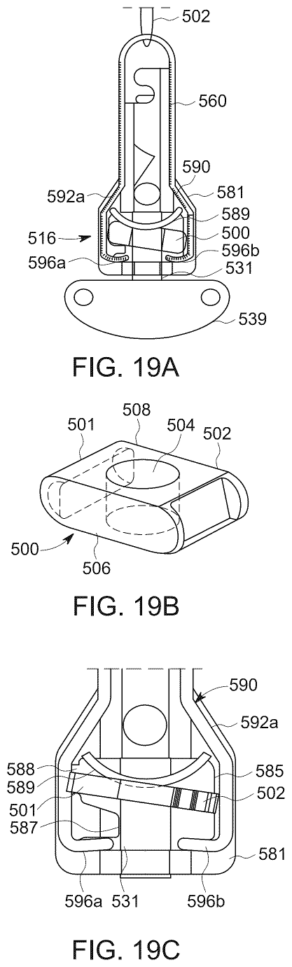

FIGS. 14, 15, and 16A-16C illustrate an embodiment of the lock 116. Lock 116 generally includes a housing 181, one or more wedging elements 180, a release harness 190, and a biasing member 189. Housing 181 may be positioned distal to coupling member 160 and may be free-floating, coupled to, or integral with coupling member 160, such as at a distal end thereof. Housing 181 may form a window 183 which may be defined at opposite sides with sloping or tapered surfaces 185 which slope inwardly toward stud 131 in a proximal to distal direction. Wedging elements 180 may be in the form of rolling elements, such as a pair of barbells, disposed on opposite sides of stud 131 and between sloping surfaces 185, as shown in FIGS. 18A and 18B. Each barbell 180 may have a pair of generally cylindrical caps 182 and a shaft 184 therebetween, as illustrated in the barbell cross-section of FIG. 16A. Barbells 180 and stud 131 are preferably comprised of cobalt chromium or stainless steel, however any suitable material may be used. Biasing member 189 may be a spring, such as a leaf spring, for example, and may be positioned at a proximal end of housing 181 between sloping surfaces 185 and proximal to barbells 180 such that spring 189 bears on barbells 180 and biases them in a distal direction. Thus, when barbells 180 are pushed distally by spring 189, they are correspondingly pushed inwardly and wedged against stud 131 by sloping surfaces 185, as illustrated by FIG. 18A, which depicts barbells 180 in a proximal and unlocked position, and FIG. 18B, which depicts barbells 180 in a distal and locked position.

As shown in FIGS. 14, 15, and 18C, release harness 190 may be in the form of a ridged wire or rod that may extend proximally from stud 131 toward a proximal end of fixation device 112 and at opposite sides thereof. In this regard, release harness 190 may form a first portion or front portion 192a and a second portion or rear portion 192b. Each of first and second portions 192a, 192b may include a crest or closed proximal end 194 through which a lock line 102 may be threaded and engaged, as described below. Release harness 190 may also form hooked distal ends 196a, 196b which may extend between first and second portions 192a, 192b and between sloping surfaces 185 and stud 131, as shown in FIGS. 18A and 18B. Thus, hooked ends 196a and 196b may be moveable proximally-distally within window 183 formed between sloped surfaces 185 and stud 131. Additionally, hooked ends 196a and 196b may be positioned distal of barbells 180 such that pulling up on harness 190 moves hooked ends 196a, 196b proximally so as to push the respective barbells 180 against the bias of spring 189 and move them to their unlocked position.

Movement of harness 190 may be performed by one or more lock line 102 which may be coupled to harness 190 by such as by threading lock line 102 through and engaging one or more of proximal ends 194 of first and second portions 192a, 192b thereof, as shown in FIGS. 14 and 15. Such lock line 102 may have a first end 102a fixedly secured to a delivery system handle 1012 of delivery system 600 and a second end 102b releasably secured to a delivery system handle 1012, as described in more detail below. In this regard, tension can be selectively applied to lock line 102 to unlock and lock the lock 116. Also, lock line 102 can be released from release harness 190 prior to, concurrently with, or after release of fixation device 112 from delivery system 600 which may be achieved by releasing the second end 102b from delivery system handle and pulling lock line 102 and its second end through shaft 111. Lock line 102 may be comprised of any suitable material, typically wire, nitinol wire, cable, suture, or thread, to name a few. In addition, lock line 102 may include a coating, such as parylene. Parylene is a vapor deposited pinhole free protective film which is conformal and biocompatible. It is inert and protects against moisture, chemicals, and electrical charge.

When an upwards force is applied to harness 190 by the lock line 102, hooked ends 196a, 196b may raise barbells 180 against spring 189, as shown in FIG. 18A. This may draw barbells 180 up along sloping surface 185 which unwedges barbells 180 from against stud 131. In this position, stud 131 is free to move. Thus, when lock line 102 is tensioned to raise or lift harness 190, lock 116 is in an unlocked position wherein stud 131 is free to move actuator 113 and therefore distal elements 120 to any desired position. Releasing tension in lock line 102 may, on the other hand, transition the lock 116 to a locked position, as shown in FIG. 18B. Thus, by releasing the upwards force on barbells 180 by hooked ends 192a, 192b, spring 189 forces barbells 180 downwards and wedges barbells 180 between a sloping surface 185 and stud 131. This restricts motion of stud 131, which in turn locks actuator 113 and therefore distal elements 120 in place. In addition, stud 131 may include one or more grooves or indentations 137 which may receive shaft 184 of each barbell 180. This may provide more rapid and positive locking by causing barbells 180 to settle in a definite position, increase the stability of lock 116 by further preventing movement of barbells 180, as well as tangible indication to the user that each barbell 180 has reached a locking position. In addition, grooves 137 may be used to indicate the relative position of distal elements 120, particularly the distance between distal elements 120. For example, each groove 137 may be positioned to correspond with a 0.5- or 1.0-mm decrease in distance between distal elements 120. As stud 131 is moved, barbells 180 may contact grooves 137, and by counting the number of grooves 137 that are felt as stud 131 is moved, the user can determine the distance between distal elements 120 and can provide the desired degree of coaptation based upon leaflet thickness, geometry, spacing, blood flow dynamics and other factors. Thus, grooves 137 may provide tactile feedback to the user.

Lock 116 allows fixation device 112 to remain in an unlocked position when attached to delivery system 600 during grasping and repositioning and then maintain a locked position when left behind as an implant. It may be appreciated, however, that lock 116 may be repeatedly locked and unlocked throughout the placement of the fixation device 112 if desired. Once the final placement is determined, lock line 102 may be removed and fixation device 112 may be left behind.

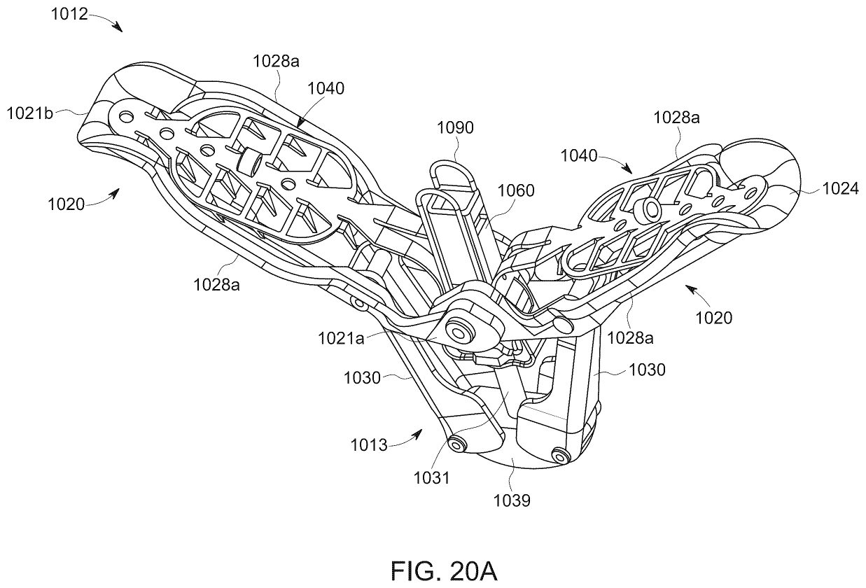

FIGS. 19A-19C depict a lock 516 according to another embodiment of the present disclosure that may be incorporated into a fixation device 112 of the disclosure. In this embodiment, lock 516 also includes a housing 581, a spring 589, a release harness 590, and a wedging element 500. However, instead of sloping surfaces 185 as present in the example of locking element 116, housing 185 may include generally parallel sidewalls 585 and may include a finger or protrusion 587 extending from one of sidewalls 585 toward stud 131, as best shown in FIG. 19C. Such finger 587 may slope in a distal direction and may define a proximal notch 588. Also, as shown in FIG. 19C, first hooked end 596a of release harness 590 may be positioned distal of finger 587.

Furthermore, wedging element may comprise a binding lever or binding plate 500. As shown in FIG. 19B, binding plate 500 may have an oblong shape that may extend lengthwise between a first end 501 and a second end 502 thereof. An aperture 504 may be formed between first and second ends 501, 502 and may extend from a top planar surface 508 through a bottom planar surface 506 of binding plate 500. Binding plate 500 may be positioned between sidewalls 406 so that stud 131 passes through aperture 504 and so that first end 501 of binding plate 500 is positioned within notch 588 proximal of finger 587, as best shown in FIG. 19C. Thus, finger 587 may be positioned between first end 501 of binding plate 500 and first hooked end 596a of released harness 590. Also, spring 589 may be positioned proximal to binding plate 500 and provide downward or distal bias thereto. Binding plate 500 and stud 131 may be comprised of any suitable material. In some embodiments, binding plate 500 may have a higher hardness than stud 131. In other embodiments, binding plate 500 may be comprised of a flexible or semi-flexible material. Such flexibility may allow slight movement of stud 131 in the proximal and distal directions, therefore allowing slight movement of distal elements 120 when lock 516 is in the locked position. This may allow fixation device 112 to adjust in response to dynamic cardiac forces.

FIGS. 19A and 19C illustrate binding plate 500 in a locked position or configuration. In this regard, spring 589 pushes binding plate 500 in a distal direction. However, because first end 501 of binding plate 501 is positioned within notch 588, axial movement of first end 501 toward a distal end of housing 581 is prohibited while axial movement of second end 502 of binding plate 500 is permitted. Thus, finger 587 obstructs first end 501 from axial movement and creates a lever type movement of binding plate 500. Moreover, finger 587 obstructs first hooked end 596a of release harness 590 from axial movement resulting in a side-to-side pivoting of release harness 590 upon tension of lock line 102. This pivoting movement correspondingly results in second hooked end 596b of release harness moving proximally and controlling movement of second end 502 of binding plate 500. As such, when an upwards force is applied to harness 590 by lock line 102, second hooked end 596b of release harness 590 raises second end 502 of plate 500 against spring 589 so that planar surfaces 506, 508 of binding plate 500 become oriented substantially perpendicular to stud 131. This aligns aperture 504 with stud 131 allowing free movement of stud 131 in the proximal-distal direction. Thus, in this state, lock 516 is unlocked wherein stud 131 is free to move actuator 113 and therefore distal elements 120 to any desired position.

Release of harness 590 by lock line 102 transitions lock 516 back to the locked position. By releasing the upwards force on second end 502 of binding plate 500, spring 589 forces second end 502 of biding plate 500 downwards, which misaligns aperture 504 relative to stud 531, and correspondingly wedges binding plate 500 against stud 131, as best shown in FIG. 19C. This arrests movement of stud 131, which in tum locks actuator 113 and therefore distal elements 120 in place. It may be appreciated that binding plate 500 may have any suitable form to function as described above. For example, plate 500 may have a variety of shapes with or without planar surfaces 506, 508 and/or the aperture 504 may be of a variety of shapes and positioned in a variety of locations, to name a few. For example, binding plate 500 may not have a through-hole, like that of aperture 504, but may rather have a notch such that binding plate 500 does not encircle stud 131 but rather partially surrounds it. Further, it may be appreciated that any number of binding plates 500 may be present. Each binding plate 500, in this regard, may provide an additional binding location which may enhance lock performance.

While the above-described nonlimiting examples of fixation device 112 may utilize a push-to-open, pull-to-close mechanism for opening and closing distal elements 120, it should be understood that a pull-to-open, push-to-close mechanism may alternatively be provided. For example, distal elements 120 may be coupled at their proximal ends to stud 131 rather than to coupling member 160, and legs 130 may be coupled at their proximal ends to coupling member 160 rather than to stud 131. In this example, when stud 131 is pushed distally relative to coupling member 160, distal elements 120 may close, while pulling on stud 131 proximally toward coupling member 160 may open distal elements 120. Regardless, the aforementioned lock examples may be configured to arrest stud to lock distal elements 120 in the desired position, as described.

It is to be understood that the fixation devices and components thereof described above are provided as examples are not to be considered as limiting to fixation devices suitable for use with other aspects of the disclosure.

Single leaflet device attachment (“SLDA”) is the most frequent device-related issue associated with TEER. Clinical observations have noted an occurrence rate of between 1% and 2% when treating the mitral valve and as high as 7% when treating the tricuspid valve. An SLDA event may occur when a leaflet of the target valve is not well secured within a fixation device, such as fixation device 112, for example. One or a combination of circumstances may result in inadequate tissue securement potentially leading to SLDA. Such circumstances may include shallow leaflet insertion due to a large gap between the leaflets, shallow leaflet insertion due to high mobility anatomy (e.g., a flail leaflet) and/or due to short leaflets, leaflet slippage during closure, irregular leaflet geometry, and poor leaflet integrity compromising the interface between the fixation device and leaflet.

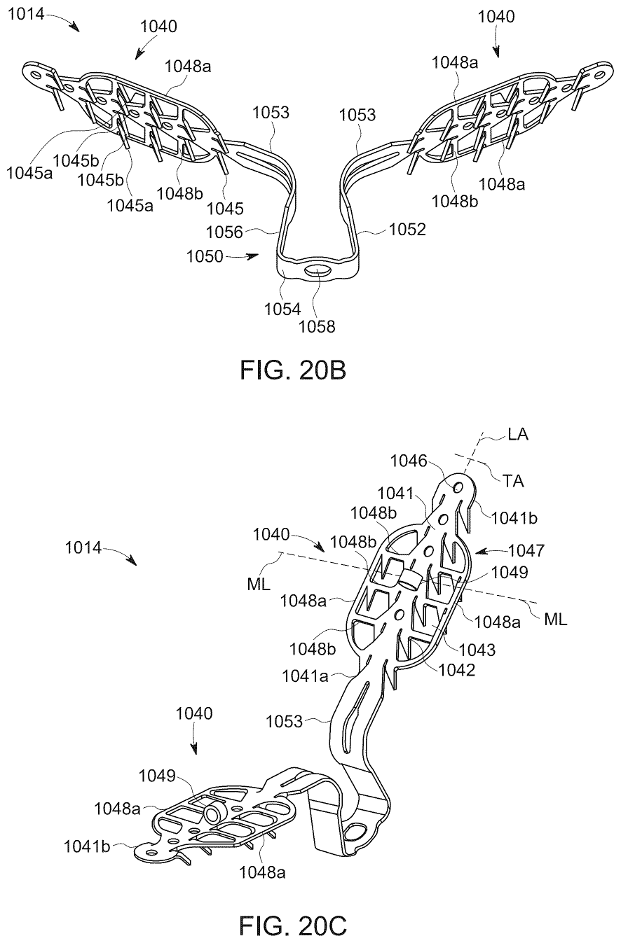

Referring now in addition to FIGS. 20A-20H, which depict a fixation device 1012 according to another embodiment of the present disclosure. Fixation device 1012 is configured to enhance tissue retention such as in circumstances of shallow leaflet insertion and/or leaflet slippage, for example. Fixation device 1012 is similar to fixation device 112 except for differences explicitly described and/or shown. Thus, like elements are accorded like reference numerals to that of fixation device 112 but within the 1000-series of numbers, meaning actuation mechanism 1013 is alike to actuation mechanism 113, coupling member 1060 is alike to coupling member 160, and so on, except for differences explicitly described and/or shown. In addition to actuation mechanism 1013 and coupling member 1060, fixation device 1012 includes a pair of distal elements or fixation elements 1020 and a gripping device 1014 that includes a pair of proximal elements or gripping elements 1040. As described in more detail below, each distal element 1020 may be a hollow distal element and each proximal element 1040 may be a gridded proximal element configured to nest within a corresponding hollow distal element 1020.

FIGS. 20B and 20C depict gripping device 1014 and proximal elements 1040 thereof. Proximal elements 1040 may each include a first end portion or fixed end 1041a and a second end portion or free end 1041b. As shown, first end portion 1041a of each proximal element 1040 may be connected to and extend from a corresponding arm bend feature 1053 which may be connected to and extend from a base section 1056. Arm bend features 1053 and base section 1050 are similar to arm bend features 153 and base section 150 of gripping device 114, which is described above. Each proximal element 1040 may include a gridded frame 1047 disposed between first end portion 1041a and second end portion 1041b. Gridded frame 1047 may include axial struts or rails 1048a, transverse struts or rails 1048b, and a plurality of frictional elements 1045 extending distally therefrom. As shown, axial struts 1048a and transverse struts 1048b intersect to form a plurality of openings or windows 1043.

In the particular embodiment depicted, proximal element 1040 may include an elongate arm 1041 extending from first end portion 1041a to second end portion 1041b and along a longitudinal axis LA. Elongate arm 1041 may be similar to elongate arm 141 in that it may include opposing side edge 1042 each forming a plurality of frictional elements 1045. Gridded frame 1047 may extend laterally outwardly from side edges 1042 of elongate arm 1041 in a transverse direction along a transverse axis TA such that gridded frame forms a pair of wing extensions extending from elongate arm 1041 comprised of axial struts 1048a (also referred to herein as “guard rails”) and transverse struts 1048b. In this regard, a plurality of transverse struts 1048b may be connected to a corresponding side edge 1042 of elongate arm 1041 and may each extend outwardly therefrom. Axial struts 1048a may also be connected to a corresponding side edge 1042 of elongate arm 1041 and extend outwardly from elongate arm 1041 and axially along axis LA such that each wing strut 1048a forms a lateral boundary of gridded frame 1047. Each axial strut 1048a may be connected to a plurality of transverse struts 1048b and may extend parallel (+/−5 degrees) to longitudinal axis LA along at least a portion of its length. Thus, each transverse strut 1048b may extend from elongate arm 1041 to a corresponding axial strut 1048a and may orthogonally intersect axial strut 1048a. Elongate arm 1041, transverse struts 1048b, and axial struts 1048a may define a plurality of openings 1043 that may extend through proximal element 1040 from a proximal side to a distal side thereof. Such openings 1043 may be rectangular or square shape, for example. However, in other embodiments, such openings 1043 may be circular, for example. As shown in FIG. 20F, elongate arm 1041 may have a first width W1 and gridded frame 1047 may have a second width W2 greater than first width W1. First width W1 may be up to about 1.5 mm, and second width W2 may be up to about 4 mm. Thus, gridded frame 1047 may more than double the width of proximal element 1040 for enhanced engagement with a leaflet. Additionally, each proximal element may have a loop 1049 at a proximal side thereof which may be configured to receive a proximal element line, such as line 101, for example. Such loop may be positioned at midline ML.

Each proximal element 1040 may include a plurality of frictional elements 1045, which may be similar to frictional elements 145 in that they may be configured for atraumatic engagement with leaflet tissue. Such frictional elements 1045 may extend from elongate arm 1041 and/or gridded frame 1047. When extending from elongate arm 1041, frictional elements 1045 may be formed from side edges 1042 of elongate arm 104 similar to elongate arm 141 of proximal element 140, for example. When extending from gridded frame 1047, frictional elements 1045 may extend from one or more transverse struts 1048b. Thus, each transverse strut 1048b may include one or more frictional elements 1045 extending distally therefrom, such as one to three frictional elements 1045, for example. As such, proximal element 1040 may have a plurality of rows of frictional elements 1045 distributed along a length of proximal element 1040. Such rows may include two to six frictional elements 1045, for example. In the depicted embodiment, proximal element 1040 may have at least two rows of frictional elements 1045 each having a plurality of frictional elements 1045 (e.g., four frictional elements) which may be located at and/or adjacent to a midline of proximal element 1045 (e.g., up to 1 mm from midpoint). In other words, each proximal element 1040 may have a length extending from first end portion 1041a to second end portion 1041b which may be bisected by a midline ML. Adjacent rows frictional elements 1045 may be located at and/or adjacent to the midline ML of the proximal element length which is facilitated by gridded frame 1047. For example, a transverse strut 1048b having one or more frictional elements 1045 may extend from the midline ML and an adjacent transverse strut 1048b having one or more frictional elements 1045 may be offset from midline ML by up to 1 mm. In another example, a pair of transverse struts 1048b may be positioned at opposite sides of midline ML (i.e., one closer to first end portion 1041 a and one closer to second end portion 1041 b) each by up to 1 mm, such as shown in FIG. 20C. Thus, proximal element 1040 may have one or more rows of frictional elements 1045 at and/or adjacent to midline ML in which each row includes a pair of outer frictional elements 1045a and a pair of inner frictional elements 1045b.

Gridded frame 1047 may increase the total number of frictional elements 1045 as compared to proximal element 140 by up to 33%, for example. Additionally, gridded frame 1047 may increase the number of frictional elements 1045 at the midline ML of proximal element 1045 as compared to proximal element 140. Pullout force tests have concluded there is a significant drop in retention force at 50% leaflet insertion and that increasing the number of frictional elements 1045 at the midline ML of proximal element 1040 enhances leaflet retention which can help mitigate SLDA caused by shallow leaflet insertion and help prevent leaflet slippage during leaflet capture. Additionally, axial struts 1048a may act as a guard along at least a portion of each proximal element 1040 which may prevent thin surrounding tissues, such as chordae tendineae CT and leaflet edges, for example, from becoming ensnared in the side edges 1042 of elongate arm 1041.

While proximal element 1040 is depicted as being connected to and extending from arm bend feature 1053 and base section 1050, which are similar to arm bend feature 153 and base section 150 of FIGS. 5A and 5C, it should be understood that proximal element 1040 can be connected to and extend from arm bend feature 260 and second arm 250 of FIGS. 6A and 6B or bend feature 260 and second arm 250′ of FIG. 6C, for example. Thus, proximal element 1040 may be adapted to couple to a central portion of a fixation device, like in fixation device 112, or adapted to couple directly to a distal element of a fixation device as has been described with respect to gripping devices 214 and 214′ of FIGS. 5A-5B and 6A-6C.

FIGS. 20D and 20E depict an exemplary distal element 1020 of fixation device 1012. Distal element 1020 may have a first end portion or fixed end 1021a and a second end portion or free end 1021b. First end portion 1021a may be configured to couple to actuation mechanism 1013 of fixation device 1012 in a manner similar to fixation device 112. Distal element 1020 may also include a hollow frame disposed between first end portion 1021a and second end portion 1021b. Hollow frame may be hollow in that it may define a cavity 1025 for receipt of gridded frame 1047 of proximal element 10410. Hollow frame 1027 (also referred to herein as strutted frame) may include a plurality of axial struts 1028a, 1028b and one or more cross-supports 1029 connected to at least a pair of axial struts 1028 a, 1028 b.

For example, in the embodiment depicted, distal element 1020 may include a pair of outer axial struts or rails 1028a and a pair of inner axial struts or rails 1028b each extending axially between first end portion 1021a and second end portion 1028b. Outer struts 1028a may have a proximal surface 1024 define a reference plane REF1 defining a proximal extent of distal element 1020, and inner struts 1028b may be offset distally from reference plane REF1, as illustrated in FIG. 20G. In other words, inner struts 1028b may be offset distally from outer struts 1028a, and outer struts 1028a may be offset laterally in the transverse direction from inner struts 1028b such that outer struts 1028a define lateral boundaries of distal element 1020. This configuration forms cavity 1025 that has a third width W3 that is greater than second width W2 of gridded frame 1047 of proximal element 1040, as illustrated in FIG. 20F. In this regard, gridded frame 1040 may nest within cavity 1025 when proximal element 1040 is lowered toward distal element 1020, as explained in more detail below. Additionally, outer struts 1028a may define a fourth width W4 of distal element 1020 which may be the maximum width of distal element 1020, as also shown in FIG. 20F. Cross support 1029 may extend between inner axial struts 1028b for structural support. Furthermore, outer and inner axial struts 1028a, 1028b may define a plurality of openings or windows 1023 extending through distal element. For example, a pair of outer openings 1023a may extend through distal element 1020 between respective outer and inner axial struts 1028a, 1028b, and an inner opening 1023b may extend through distal element 1020 between inner axial struts 1028b, as shown in FIGS. 20D and 20E. Inner and outer openings 1023a, 1023b may be elongate openings 1028a, 1028b extending along at least a portion of a length of hollow frame 1027.

Although distal element 1020 is shown having a hollow frame 1027 with a plurality of axial struts 1028a, 1028b offset from each other and defining a plurality of openings 1023a, 1023b, in some embodiments, hollow frame 1027 may not have separate axial struts 1028a, 1028b, but may have a continuous structure extending axially between first and second end portions 1021a, 1021b and laterally to define cavity 1025 and fourth width W4. However, as described below, a hollow frame 1027 with axial supports 1028a, 1028b may provide additional benefits in terms of leaflet fixation.

FIGS. 20F-20H illustrate one example of leaflet capture between distal and proximal elements 1020, 1040 fixation device 1012. In operation, distal and proximal elements 1020, 1040 are moveable relative to each other between various different positions in a manner similar to fixation device 112, as described above. When a proximal element 1040 is lowered toward corresponding distal element 1040, such as when distal element 1040 is in the open position for capturing tissue, for example, proximal element 1020 may nest within distal element 1040. In this regard, second end portion 1041b of proximal element 1021b may nest within second end portion 1021b of distal element 1020, and gridded frame 1047 of proximal element may nest within cavity 1028 of hollow frame 1027, as shown in FIG. 20F. When gridded frame 1047 is disposed within cavity 1025, at least one inner strut 1028b may be received between respective outer and inner frictional elements 1045a, 1045b of a row of frictional elements 1045 such that outer frictional element 1045a is positioned on one side of inner strut 1028b and inner frictional element 1045b is positioned on another side of inner strut 1028b, as illustrated in FIGS. 20F-20H. In other words, when gridded frame 1040 nests within cavity 1025, an outer friction element 1045a may extend into outer opening 1023a, and an inner frictional element 1045b may extend into inner opening 1043b. This creates an over-under configuration and a tortuous path for a leaflet as it extends across fourth width W4. Thus, when a leaflet LF is captured between corresponding distal and proximal elements 1020, 1040, as shown in FIG. 20H, leaflet LF may fold over outer and inner struts 1028a, 1028b and under outer and inner frictional elements 1045a, 1045b which further increases friction applied to leaflet LF by distal and proximal elements 1020, 1040 as compared to frictional elements 1045 alone. As such, the over-under configuration of distal and proximal elements 1020, 1040 enhances fixation of leaflet LF particularly at a 50% insertion depth. Although the over-under configuration is shown with respect to pairs of outer and inner friction elements 1045a, 1045b, an over-under configuration may be formed in embodiments in which only inner or only outer frictional elements 1045a, 1045b are provided. However, the over-under configuration utilizing both inner and outer frictional elements 1045a, 1045b provides additional friction relative to just inner and outer frictional elements 1045a, 1045b alone.

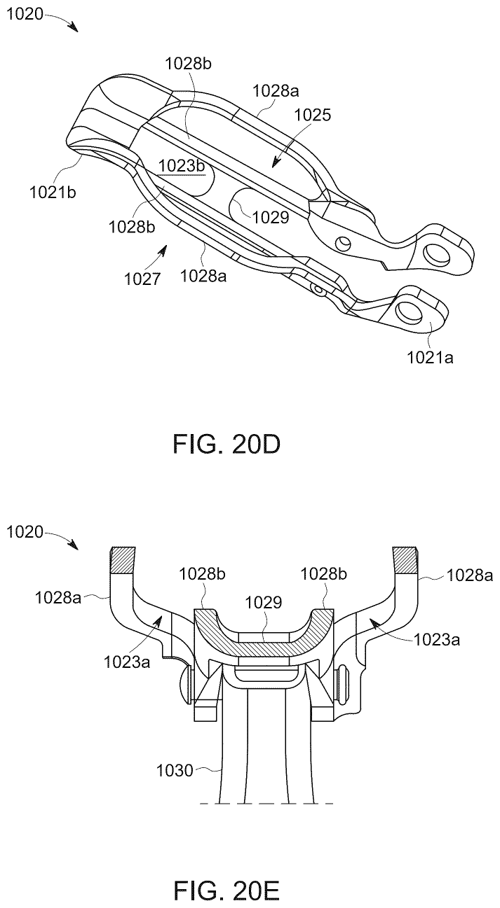

Referring now in addition to FIG. 21, which depicts a proximal element 1140 according to another embodiment of the present disclosure, which may be incorporated into any of the fixation devices of the disclosure and include any of the characteristics and features of other proximal elements disclosed except as explicitly stated. For example, proximal element 1140 may be like proximal element 1040 in that it may include a gridded frame 1147 with transverse members 1148b and at least one row of inner and outer frictional elements 1145a, 1145b for over-under engagement with distal element 1020, as described above. However, unlike proximal element 1040, proximal element 1140 may be curved or bent in a transverse plane which is parallel to or coplanar with transverse axis TA such that an upper surface or proximal surface 1144a of proximal element 1140 is concave and a lower surface or distal surface 1144b is convex. In the embodiment depicted, transverse members 1148b extending from elongate arm 1141 may be bent proximally such that outer frictional elements 1145a disposed on transverse members 1148b may point outwardly away from inner frictional elements 1145b. In some embodiments, elongate arm 1141 itself may not be bent such that inner frictional elements 1145b disposed on elongate arm 1141 may extend distally and parallel (+/−5 degrees) to each other, as shown in FIG. 21. However, in other embodiments elongate arm 1141 may also be curved or bent proximally so that inner frictional elements 1145b may also point outwardly away from each other.

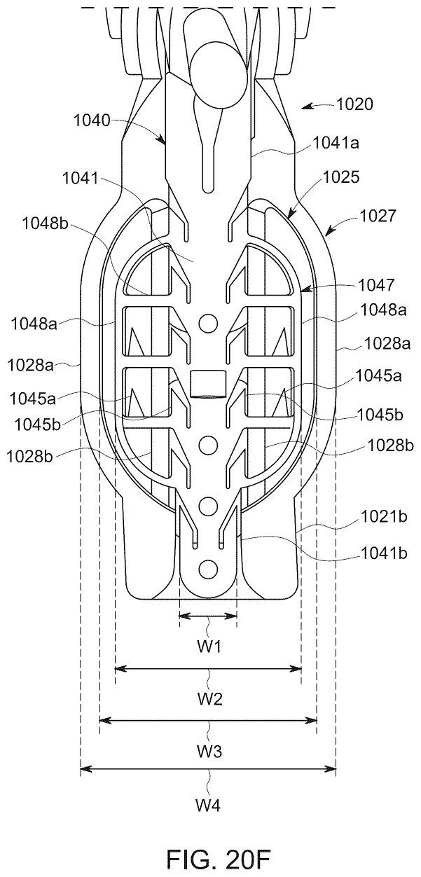

Referring now in addition to FIG. 22, which depicts a proximal element 1240 according to a further embodiment of the present disclosure, which may be incorporated into any of the fixation devices of the disclosure and include any of the characteristics and features of other proximal elements disclosed except as explicitly stated. For example, proximal element 1240 may be like proximal element 1040 in that it may include a gridded frame 1247 with transverse members 1248b and at least one row of inner and outer frictional elements 1245a, 1245b for over-under engagement with distal element 1020, as described above. However, unlike proximal element 1040, proximal element 1240 may be curved or bent in a transverse plane which is parallel to or coplanar with transverse axis TA such that an upper surface or proximal surface 1244a of proximal element 1240 is convex and a lower surface or distal surface 1244b is concave. In the embodiment depicted, transverse members 1248b extending from elongate arm 1241 may be bent distally such that outer frictional elements 1245a disposed on transverse members 1248b may point inwardly toward inner frictional elements 1245b. In some embodiments, elongate arm 1241 itself may not be bent such that inner frictional elements 1245b disposed on elongate arm 1241 may extend distally and parallel to each other, as shown in FIG. 22. However, in other embodiments elongate arm 1241 may also be curved or bent distally so that inner frictional elements 1245b may also point outwardly toward each other.

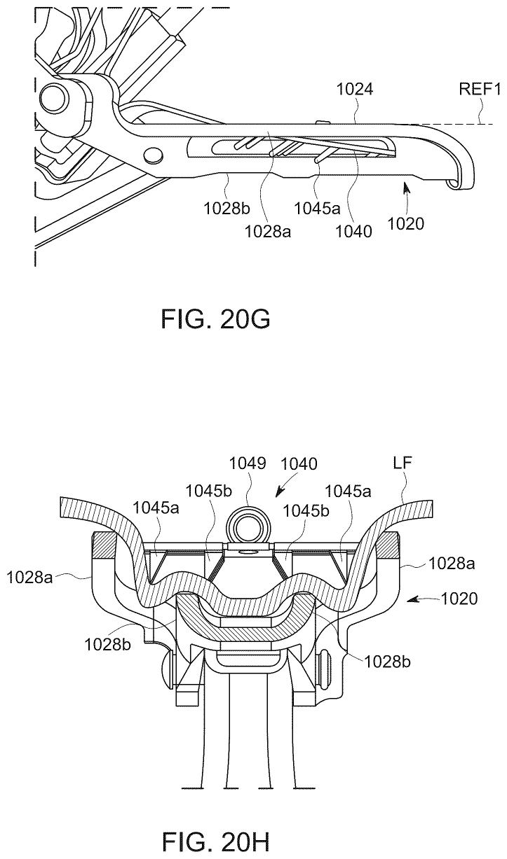

Referring now in addition to FIG. 23, which depicts a proximal element 1340 according to another embodiment of the present disclosure, which may be incorporated into any of the fixation devices of the disclosure and include any of the characteristics and features of other proximal elements disclosed except as explicitly stated. For example, proximal element 1340 may be similar to proximal element 140 or proximal element 1040 with the exception that frictional elements 1345 of proximal element may be curved in a direction away from free end 1341b and toward fixed end 1341a. Such curved frictional elements 1345 may help with retraction from a leaflet when proximal element 1340 is raised to attempt a recapture and may provide enhanced grip when engaged to a leaflet.

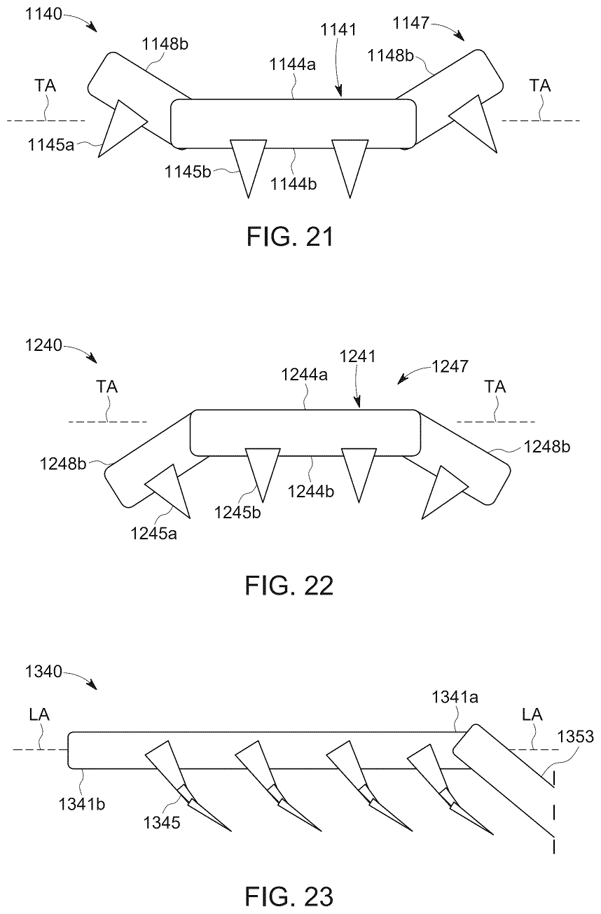

Referring now in addition to FIG. 24, which depicts a proximal element 1440 according to a further embodiment of the present disclosure, which may be incorporated into any of the fixation devices of the disclosure and include any of the characteristics and features of other proximal elements disclosed except as explicitly stated. Proximal element 1440 may be similar to proximal element 140 or proximal element 1040 with the exception that each frictional element 1445 along its length may be angled relative to a distal surface 1444b of proximal element 1440 at a different angle than a longitudinally adjacent frictional element 1445. For example, a first proximal element 1445a may be angled relative to distal surface 1444b at a first angle Θ1, and a second proximal element 1445b offset from first frictional element 1445a along the length of proximal element 1440 may be angled relative to distal surface 1444b at a second angle Θ2. First angle Θ1 differs from second angle Θ2. More specifically first frictional element 1445a may be closer to free end 1441b of proximal element 1440 than second frictional element 1445b, and first and second frictional elements 1445a, 1445b may be angled in a direction toward fixed end 1441a such that first and second angles Θ1, Θ2 are acute angles and first angle Θ1 is greater than second angle Θ2. First frictional element 1445a may be in a row with one or more first frictional elements 1445a that may extend at the same first angle Θ1, and second frictional element 1445b may be in a row with one or more second frictional elements 1445b that may extend at the same second angle Θ2. Where additional rows of frictional elements 1445 are provided, the angles at which the frictional elements 1445 in each row extend from distal surface 1444b may become incrementally smaller the closer the row is to fixed end 1441a. Additionally, the lengths at which each frictional element 1445 protrudes from distal surface 1444b may become incrementally smaller from the first row (i.e., the one closest to fixed end 1441b) to the nth row (i.e., the row closest to fixed end 1441a). The differential angles and/or differential lengths between each row of frictional elements 1445 may help create a ratcheting effect as proximal element 1440 engages a leaflet such that the leaflet is progressively tensioned with each successive row of frictional elements 1445 engaging the leaflet.

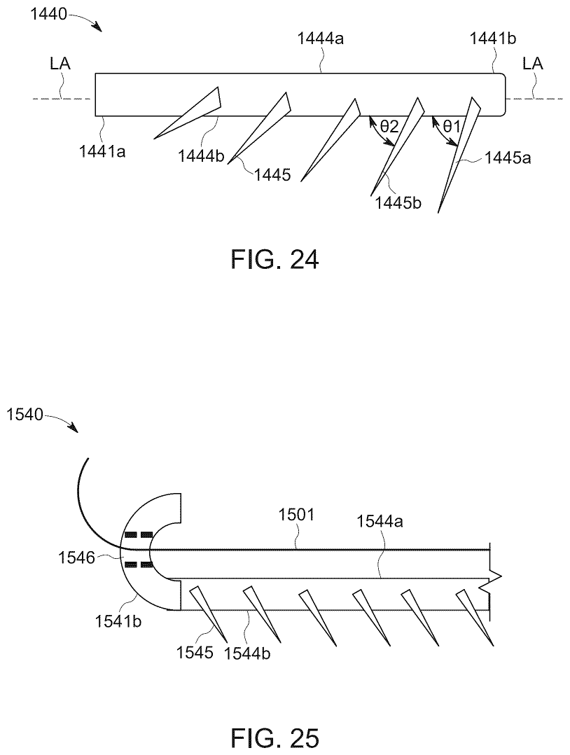

Referring now in addition to FIG. 25, which depicts a proximal element 1540 according to another embodiment of the present disclosure, which may be incorporated into any of the fixation devices of the disclosure and include any of the characteristics and features of other proximal elements disclosed except as explicitly stated. For example, proximal element 1540 may be similar to proximal element 140 or proximal element 1040 with the exception that each frictional element 1545 along the length of proximal element 1540 may be angled relative to a distal surface 1544b at the same angle and in a direction away from free end 1541b. Only one frictional element 1545 is shown for ease of illustration. It is to be understood that each frictional element 1545 may be identically configured or may be differently configured in accordance with the disclosure. Additionally, free end 1541b of proximal element 1540 may be rolled upwardly or proximally such that an opening 1546 extending therethrough may receive of a proximal element line, such as line 101, for example. Rolling free end 1541b of proximal element 1540 in this manner may help prevent a leaflet from becoming caught behind proximal element 1540. As indicated, it should be understood that this rolled-end configuration may be utilized in all proximal elements described herein.

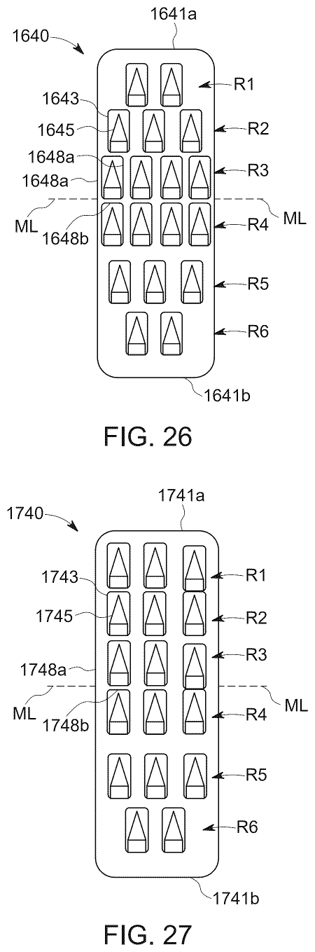

Referring now in addition to FIGS. 26-29, which depict alternative gridded proximal element embodiments all of which illustrate a variety of frictional element arrangements which may be applicable to gridded proximal element.

FIG. 26 depicts another example of a proximal element 1640, which may be incorporated into any of the fixation devices of the disclosure and include any of the characteristics and features of other proximal elements disclosed except as explicitly stated. Proximal element 1640 includes rows of frictional elements 1645 closer to fixed end 1641a and free end 1641b have fewer frictional elements 1645 than rows of frictional elements 1645 closer to a midline ML of proximal element 1640. For example, proximal element 1640 may have six rows of proximal elements 1645 such that a first row R1 is closest to fixed end 1641a, a sixth row R6 is closest to free end 1641b, and third and fourth rows R3, R4 are adjacent to and closest to midline ML. First row R1 and sixth row R6 may each include two frictional elements 1645, and third and fourth rows R3, R4 may each include four frictional elements 1645, for example. Additionally, a second row R2 may be located between first and third rows R1, R3, and a fourth row R4 may be located between fourth and sixth rows R4, R6. Second row R2 and fourth row R4 may each include three frictional elements 1645, for example. Thus, proximal element 1640 exemplifies a frictional element arrangement in which each successive row extending from midline ML may include one less frictional element 1645 than an adjacent row. Other embodiments reflecting a similar configuration may instead include one frictional element 1645 at rows R1 and R6, two frictional elements 1645 at rows R2 and R4, and three frictional elements at rows R3 and R4, for example. In further embodiments in which less than six rows of frictional elements 1645 are provided, such as four rows, for example, the end rows may have two or three frictional elements 1645, while the middle rows closest to midline ML may have three to four frictional elements 1645, for example. Thus, as shown in FIG. 26, the increased frictional element density at midline ML enhances fixation for leaflets inserted to a 50% leaflet insertion depth which may help mitigate incidence of SLDA.

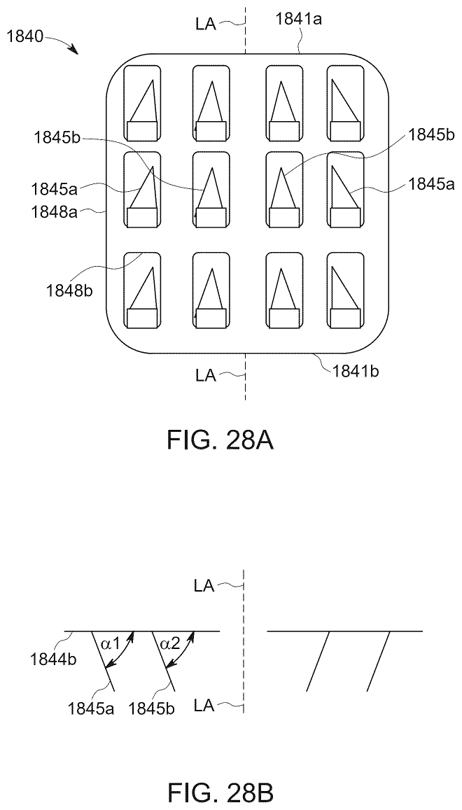

Referring now in addition to FIG. 27, which depicts an additional example of a proximal element 1740, which may be incorporated into any of the fixation devices of the disclosure and include any of the characteristics and features of other proximal elements disclosed except as explicitly stated. Proximal element 1740 includes rows of frictional elements 1745 (only one of which is referenced for ease of illustration) closer to fixed end 1741a and a midline ML of proximal element 1740 may have more proximal elements 1745 than that closest to free end 1741b. For example, proximal element 1740 may have six rows of proximal elements 1745 with a first row R1 being closest to fixed end 1741a and a sixth row R6 being closest to free end 1741b. In the embodiment depicted first row R1, second row R2, third row R3, fourth row R4, and fifth row R5 may each include three frictional elements 1745, while sixth row R6 may include two frictional elements 1745. Third and fourth rows R3, R4 may be closest to midline ML. In other embodiments reflecting a similar configuration, rows R1-R5 may each include four frictional elements 1745, and sixth row R6 may have two to three frictional elements 1745. In further embodiments, fifth and sixth rows R5, R6 may have one to two frictional elements 1745 while rows R1-R4 may each include three to four frictional elements 1745. Thus, as shown in FIG. 27, the increased density of frictional elements 1745 at midline ML helps enhance fixation of leaflets at a 50% insertion depth. Additionally, leaflets tend to have thicker leaflet tissue at their free edge and thinner tissue closer to the valve annulus. The increased density of frictional elements 1745 adjacent fixed end 1741a may help capture this thicker tissue at a full depth of leaflet insertion, while the lower density rows adjacent to free end 1741b may help protect the thinner leaflet tissue.

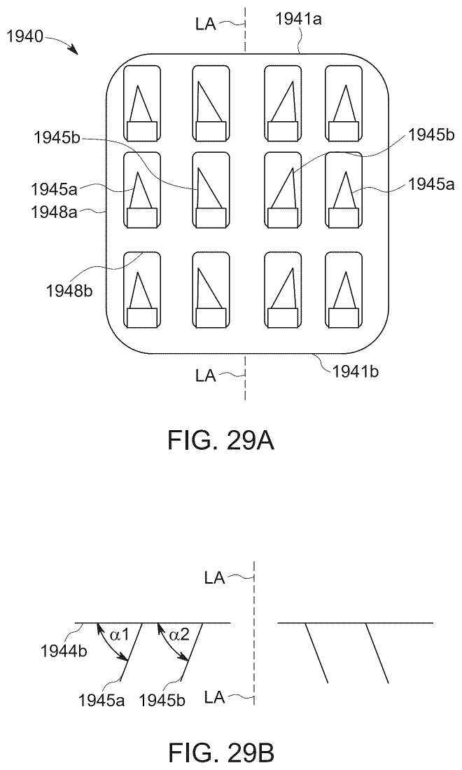

Referring now in addition to FIGS. 28A and 28B, which depict another example of a proximal element 1840, which may be incorporated into any of the fixation devices of the disclosure and include any of the characteristics and features of other proximal elements disclosed except as explicitly stated. Proximal element 1840 includes frictional elements 1845 (only one of which is referenced for ease of illustration) in each row of frictional elements 1845 tilt inwardly toward a longitudinal axis LA of proximal element 1840. For example, a row of frictional elements 1845 may include a pair of outer frictional elements 1845a tilting inwardly at a first tilt angle α1 and a pair of inner frictional elements 1845b tilting inwardly at a second tilt angle α2. Tilt angles α1, α2 are defined between an axis of its respective frictional element 1845 and a distal surface 1844b of proximal element 1840, as shown in FIG. 28B. First and second tilt angles α1, α2 may be equal, for example. In another example, first and second tilt angles α1, α2 may differ such that second tilt angle α2 is greater than first tilt angle α1. In a further example, first tilt angle α1 is greater than second tilt angle α2. The inward tilt of frictional elements 1845a, 1845b may help distribute contact forces on leaflets particularly in the over-under configuration with distal element 1020, as described above.

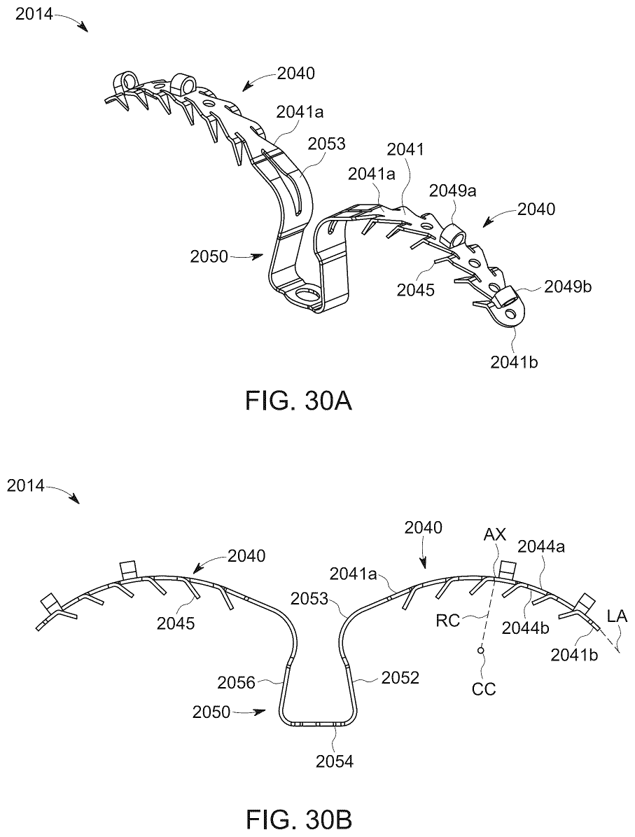

Referring now in addition to FIGS. 29A and 29B, which depict yet another example of a proximal element 1940, which may be incorporated into any of the fixation devices of the disclosure and include any of the characteristics and features of other proximal elements disclosed except as explicitly stated. Proximal element 1940 in which frictional elements 1945 (only one of which is referenced for ease of illustration) in each row of frictional elements tilt outwardly toward a longitudinal axis LA of proximal element 1940. For example, a row of frictional elements 1945 may include a pair of outer frictional elements 1945a tilting outwardly at a first tilt angle α1 and a pair of inner frictional elements 1945b tilting outwardly at a second tilt angle α2. Tilt angles α1, α2 are defined between an axis of its respective frictional element 1945 and a distal surface of proximal element 1944b, as shown in FIG. 29B. First and second tilt angles α1, α2 may be equal, for example. In another example, first and second tilt angles α1, α2 may differ such that second tilt angle α2 is greater than first tilt angle α1. In a further example, first tilt angle α1 is greater than second tilt angle α2. The outward tilt of frictional elements 1945a, 1945b may help distribute contact forces on leaflets particularly in the over-under configuration with distal element 1020, as described above.

Additional proximal element embodiments have a combination of the embodiments of FIGS. 28A-28B and 29A-29B and such proximal elements can be incorporated into any fixation devices of the disclosure. For example, one or more outer frictional elements may tilt inwardly like frictional elements 1845a, and one or more inner frictional elements may tilt outwardly like frictional elements 1945b. In another example, one or more outer frictional elements may tilt outwardly like frictional elements 1945a, and one or more inner frictional elements may tilt inwardly like frictional elements 1845b.

Although the frictional element arrangements of embodiments of FIGS. 26-29B as described above may be applicable to gridded proximal element 1040, such embodiments also illustrate alternative axial and transverse strut configurations. For example, gridded proximal element 1040 is described above as having an elongate arm 1041 like that of proximal element 140 with a gridded frame 1047 extending outwardly therefrom. However, as shown in FIGS. 26-29, a proximal element may not have an elongate arm like that of proximal element 140 and may instead be comprised of axial struts and transverse struts that together define second width W2. For example, FIG. 26 depicts proximal element 1640 with a plurality of axial struts 1648a offset from one other and arranged parallel (+/−5 degrees) to one another at incremental intervals along a width of proximal element 1640. Similarly, a plurality of transverse struts 1648b (only one of which is referenced for ease of illustration) may be offset from one another and arranged parallel (+/−5 degrees) to one another at incremental intervals along a length of proximal element 1640 such that transverse struts 1648b and axial struts 1648a intersect to define a plurality of openings 1643 (only one of which is referenced for ease of illustration). Frictional elements 1645 (only one of which is referenced for ease of illustration), may extend from transverse struts 1648a (only a few of which is referenced for ease of illustration). The proximal elements of FIGS. 27-29B may have a gridded frame configuration.

Leaflet capture represents one of the most decisive aspects of a TEER procedure for achieving a successful outcome. In some cases, gaps between leaflets are so large that grasping and capturing the leaflets while the fixation device, such as fixation device 112, is positioned at a common capture angle (e.g., 120 degrees) is not possible. Large leaflet gaps are particularly prevalent in patients with functional valve disease wherein the annulus, atrium, and/or ventricle are significantly dilated. In such circumstances, distal elements, such as distal elements 120, may be opened to their maximum width (e.g., 180 degrees) to help span the gap. However, when attempting to capture leaflets at these large capture angles, proximal elements, such as proximal elements 140, may not lower sufficiently to adequately secure the leaflets. In these cases, the operator may be required to employ multiple fixation devices to “zipper” a line of coaptation LOC or may need to resort to independent leaflet capture, which can be technically challenging.

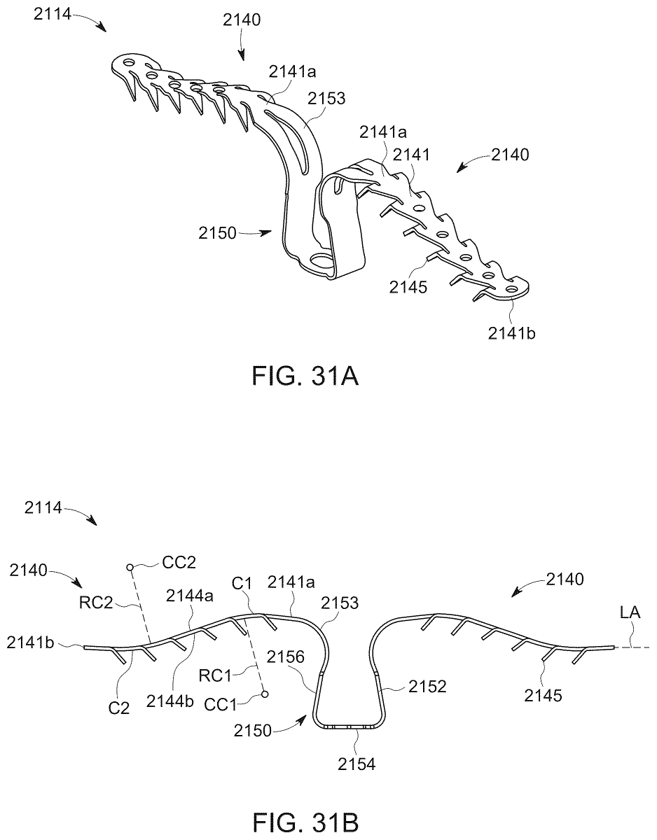

Referring now in addition to FIGS. 30A and 30B, which depict a gripping device 2014 according to another embodiment of the present disclosure, which may be incorporated into any of the fixation devices of the disclosure. Gripping device 2014 is configured to enhance tissue retention, such as in the presence of particularly large leaflet gaps, short leaflets, and highly mobile leaflets, for example. In this regard, gripping device 2014 is configured to engage tissue at greater than normal fixation device angles (e.g., angles greater than 120 degrees and up to 180 degrees). Also, as described in more detail below, gripping device 2014 may also help prevent shallow leaflet insertion.

Gripping device 2014 is similar to gripping device 114 except for differences explicitly described and/or shown and may include any features or characteristics of any other gripping device of the disclosure. Thus, like elements are accorded like reference numerals to that of gripping device 114 but within the 2000-series of numbers, meaning base section 2050 is alike to base section 150, arm bend features 2053 are alike to arm bend features 153, and so on, except for differences explicitly described and/or shown. In addition to base section 2050 and arm bend features 2053, gripping device 2014 generally includes a pair of proximal elements 2040.

As shown, each proximal element 2040 may be in the form of elongate arm 2041 that has a first end portion or fixed end 2041a that may be connected to an arm bend feature 2053, a second end portion or free end 2041b disposed opposite fixed end 2041a, and a plurality of frictional elements 2045 (only one of which is referenced for ease of illustration) located between fixed end 2041a and free end 2041b. Arm 2041 may be bent or curved along its length from fixed end 2041a to free end 2041b. More specifically, each proximal element 2040 may be curved in a plane parallel to a longitudinal axis LA thereof and may form a single curvature that is concave in a distal direction and convex in a proximal direction. In other words, a bottom or distal surface 2044b of each proximal element 2040 may be concavely curved, and an upper surface or proximal surface 2044a of each proximal element 2040 may be convexly curved. Such curvature defines a center of curvature CC and a radius curvature RC extending between bottom surface 2044b and center of curvature CC. The center of curvature CC is located distal of each proximal element 2044b. Thus, each proximal element 2040 may be referred to as a single-curve proximal element 2040.

In this single-curve configuration, an apex AX is formed at a midpoint of the elongate arm 2041 which may be about equidistant (+/−5%) between the first end portion 2041a and second end portion 2041b. Such apex AX is the most proximal point of proximal element 2040 when gripping device 2014 is in an unstressed state. When implemented in a fixation device, such as fixation device 112, it may be desirable to raise and lower proximal element 2040 as described above with respect to gripping device 114. To help facilitate raising and lowering of proximal element 2040, each proximal element 2040 may include a first loop 2049a and a second loop 2049b which may each be configured to receive a proximal element line, such as proximal element line 101, for actuation of proximal element 2040. First loop 2049a may be positioned at or near apex AX and second loop 2049b may be positioned at or near free end 2041b. This configuration of loops 2049a, 2049b may help straighten proximal element 2040 particularly when in the raised position to help maintain a low profile when gripping device 2014 transported through a catheter and at the initial stage of leaflet insertion and capture.

Like gripping device 114, gripping device 2014 may be made of a flexible material, such as a shape-memory-metal, such as nitinol, for example. The single curvature of proximal element 2040 predisposes free end 2041b at a lower elevation than free end 141b of proximal element 120 when in an unstressed state. Thus, as proximal element 2040 is lowered onto a leaflet, free end 2041b makes contact with a leaflet first and sooner than would free end 141b of proximal element 140 and with increased leaflet retention force than with proximal element 140 thereby increasing the likelihood of securing the leaflet in the event of shallow leaflet insertion. As such, each proximal element 2040 is configured to make contact with a leaflet from outside-in when lowered toward a leaflet which may help to better obtain full leaflet insertion at any grasping angle including large grasping angles (e.g., greater than 120 degrees and up to 180 degrees). This may improve the ease of leaflet grasping particularly for valves with large gaps. Additionally, gripping device 2014 may help increase annulus remodeling. When a fixation device is closed on a dilated/diseased valve, tension is applied through the captured leaflets, which beneficially acts on the valve annulus. This tension may reduce the valve diameter (i.e., anterior-posterior dimension for the mitral valve and septal-lateral dimension for the tricuspid valve) and may help restore a diseased (dilated) valve back to a more healthy and physiologic geometry. Grasping leaflets at large grasping angles (e.g., greater than 120 degrees and up to 180 degrees) both maximizes leaflet insertion and also allows the operator to apply even more beneficial tension to the valve annulus when closing the fixation device. Furthermore, capturing a leaflet outside-in with gripping device 2014 may increase leaflet insertion by performing an “inchworm” technique. By closing and opening a fixation device, the curved configuration of proximal element 2040 straightens and spreads out as it is lowered onto a leaflet to reach out for more leaflet than was initially inserted. By repeatedly opening and closing a fixation device, it may be possible to repeatedly curve and straighten proximal element 2040 thereby pulling in more leaflet than was originally inserted.

Gripping device 2014 may be utilized in conjunction with distal elements 120 of fixation device 112, as an example. Additionally, while proximal element 2040 is depicted as being connected to and extending from arm bend feature 2053 and base section 2050, which are similar to arm bend feature 153 and base section 150 of FIGS. 5A and 5C, it should be understood that proximal element 2040 can be connected to and extend from arm bend feature 260 and second arm 250 of FIGS. 6A and 6B or bend feature 260 and second arm 250′ of FIG. 6C, for example. Thus, proximal element 2040 may be adapted to couple to a central portion of a fixation device, like in fixation device 112, or adapted to couple directly to a distal element of a fixation device as has been described with respect to gripping devices 214 and 214′ of FIGS. 5A-5B and 6A-6C. Furthermore, although gripping device 2014 has been depicted with both proximal elements 2040 being curved along their respective lengths, it should be understood that in some embodiments, one proximal element may be straight, like proximal element 140, and another proximal element may be curved, like proximal element 2040. This may help discern between proximal elements using medical imaging during a TEER procedure and to direct curved proximal element 2040 to a particularly troublesome leaflet, such as a flail leaflet, for example.

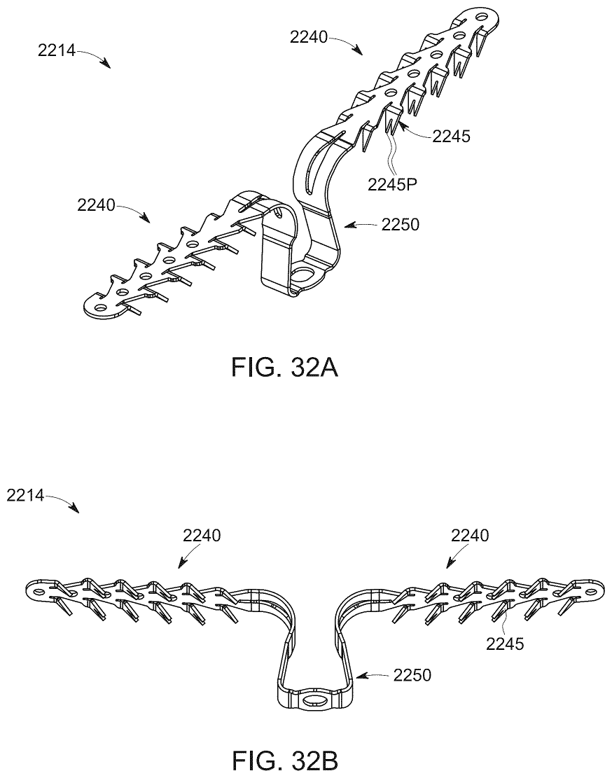

Referring now in addition to FIGS. 31A and 31B, which depict one example of a gripping device 2114 according to another embodiment of the present disclosure, which may be incorporated into any of the fixation devices of the disclosure. Gripping device 2114 is also configured to enhance tissue retention, such as in the presence of particularly large leaflet gaps, for example. In this regard, gripping device 2114 is configured to engage tissue at greater than normal fixation device angles (e.g., angles of up to 180 degrees). Also, as described in more detail below, gripping device 2114 may also help prevent shallow leaflet insertion.

Gripping device 2114 is similar to gripping device 114 except for differences explicitly described and/or shown and may include any features or characteristics of any other gripping device of the disclosure. Thus, like elements are accorded like reference numerals to that of gripping device 114 but within the 2100-series of numbers, meaning base section 2150 is alike to base section 150, arm bend features 2153 are alike to arm bend features 153, and so on, except for differences explicitly described and/or shown. In addition to base section 2150 and arm bend features 2153, gripping device 2114 generally includes a pair of proximal elements 2140.

As shown, each proximal element 2140 may be in the form of elongate arm 2141 that has a first end portion or fixed end 2141a that may be connected to an arm bend feature 2153, a second end portion or free end 2141b disposed opposite fixed end 2141a, and a plurality of frictional elements 2145 (only one of which is referenced for ease of illustration)located between fixed end 2141a and free end 2141b. Arm 2141 may be bent or curved at multiple locations along its length from fixed end 2141a to free end 2141b. More specifically, each proximal element 2141 may have a first curve C1 and a second curve C2 where first curve C1 and second curve C2 are curved in a plane parallel to a longitudinal axis LA of proximal element 2140. As shown, first curve C1 may extend from a bend feature 2153 and may be concave in a distal direction, and second curve C2 may extend from first curve C1 to free end 2141b and may be concave in a proximal direction. In other words, each proximal element 2140 has a proximal surface or upper surface 2144a and a distal surface or lower surface 2144b. Distal surface 2144b may be concave at first curve C1, and proximal surface 2144a may be concave at second curve C2. As such, first curve C1 may define a first center of curvature CC1 disposed distal to proximal element 2140 and a first radius of curvature RC1 extending between distal surface 2144b and first center of curvature CC1. Second curve C2 may define a second center of curvature CC2 disposed proximal to proximal element 2140 and a second radius of curvature CR2 extending between proximal surface 2144a and second center of curvature CC2. As shown in FIG. 31B, such multi-curvature of proximal element 2140 may form a sinuous, undulating (i.e., S) shape between fixed end 2141a and free end 2141b. Thus, each proximal element 2140 may be referred to as a multi-curve proximal element 2141. Although not depicted, each proximal element 2140 may have multiple suture loops for engagement with a proximal element line similar to that of proximal element 2040 to help facilitate a straightened profile in a raised position or prevent adverse gripper interactions with leaflets.

Like gripping device 114, gripping device 2140 may be made of a flexible material, such as a shape-memory-metal, such as nitinol, for example. The multi-curve configuration of proximal element 2141 predisposes free end 2141b at a lower elevation than free end 141b of proximal element 120 when in an unstressed state. Thus, as proximal element 2141 is lowered onto a leaflet, free end 2141b makes contact with a leaflet first and sooner than would free end 141b of proximal element 140. As such, each proximal element 2140 is configured to make contact with a leaflet from outside-in when lowered toward a leaflet which may help to better obtain full leaflet insertion at any grasping angle including large grasping angles (e.g., up to 180 degrees). Thus, gripping device 2114 may realize similar benefits as those described above with respect to gripping device 2014. However, because of the multiple curvatures of proximal element 2140, free end 2141b may not be disposed at as low of an elevation as free end 2041b when gripping device 2114 is in an unstressed state. On the other hand, at least because second curvature C2 curves proximally, free end 2141b may more readily present frictional elements 21456 to a leaflet for initial engagement to help prevent leaflets from slipping out of the fixation device. Thus, the curves of grippers 2140 can increase leaflet retention force at the outermost frictional elements 2145 which increases the likelihood of securing leaflets with partial leaflet insertion and allow grippers 2140 to engage tissue at large angles of fixation elements 120 (e.g., greater than 120 degrees up to 180 degrees).

Gripping device 2114 may be utilized in conjunction with distal elements 120 of fixation device 112, as an example. Additionally, while proximal element 2140 is depicted as being connected to and extending from arm bend feature 2153 and base section 2150, which are similar to arm bend feature 153 and base section 150 of FIGS. 5A and 5C, it should be understood that proximal element 2140 can be connected to and extend from arm bend feature 260 and second arm 250 of FIGS. 6A and 6B or bend feature 260 and second arm 250′ of FIG. 6C, for example. Thus, proximal element 2140 may be adapted to couple to a central portion of a fixation device, like in fixation device 112, or adapted to couple directly to a distal element of a fixation device as has been described with respect to gripping devices 214 and 214′ of FIGS. 5A-5B and 6A-6C. Furthermore, although gripping device 2141 has been depicted with both proximal elements 2141 having multiple curves along their respective lengths, it should be understood that in some embodiments, one proximal element may be straight, like proximal element 140, and another proximal element may be curved, like proximal element 2141. This may help discern between proximal elements using medical imaging during a TEER procedure and to direct curved proximal element 2141 to a particularly troublesome leaflet, such as a flail leaflet, for example.

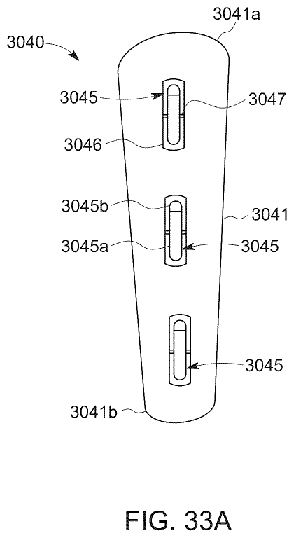

Referring now in addition to FIGS. 32A and 32B, which depict one example of a gripping device 2214 according to another embodiment of the present disclosure, which may be incorporated into any fixation device of the disclosure. In one example, gripping device 2114 is similar to gripping device 114 with the differences being that each proximal element 2240 includes a greater number of frictional elements 2245 (e.g., six rows, only one frictional element is labeled for ease of illustration) and at least some of frictional elements 2245 include multiple prongs 2245P which greatly increases the friction applied to a leaflet. In various examples, one or more prongs 2245P may include a tapered and/or pointed shape.

Each of the aforementioned proximal elements 140, 1040-2140 may be covered with a covering, similar to covering 117, such that frictional elements 145, 1045-2145 thereof extend through the covering. However, in some embodiments, such proximal elements 140, 1040-2140 may only be partially covered by a covering such that only some of frictional elements 145, 1045-2145 extend through the covering. Such partial covering may make the frictional elements 145, 1045-2145 that are not covered act like longer frictional elements as compared to those that extend through a covering.

TEER therapy is a minimally invasive interventional procedure that aims to approximate cardiac valvular leaflets to reduce valvular regurgitation particularly in the mitral and triscupid valves. One of the challenges of this procedure is ensuring that the valve leaflets are adequately inserted into the fixation device, such as fixation device 112, for example, so that when the fixation device is deployed, it remains secured to the tissue and sufficiently reduces regurgitation. Current TEER techniques typically rely on echocardiography and fluoroscopy for visualization which typically capture views of the target valve at fixed angles which are commonly oriented orthogonal to each other. Such visualization modalities, however, have inherent limitations that present challenges making leaflet capture and grasping one of, if not the most, challenging aspects of a TEER procedure. In particular, technique and technological limitations make it difficult to visually observe the leaflets within the fixation device during and after deployment so that the surgeon cannot directly observe how much leaflet is inserted into the fixation device and the quality of the capture. Thus, surgeons typically assess leaflet capture through circumstantial observations that are heavily reliant on surgeon experience. For example, an operator may observe changes in movements of the leaflets and reductions in backflow via color doppler to estimate the adequacy of leaflet capture. Thus, it may not be apparent that shallow insertion or other potential failure modes are present until SLDA or complete detachment (i.e., implant embolization) occurs.

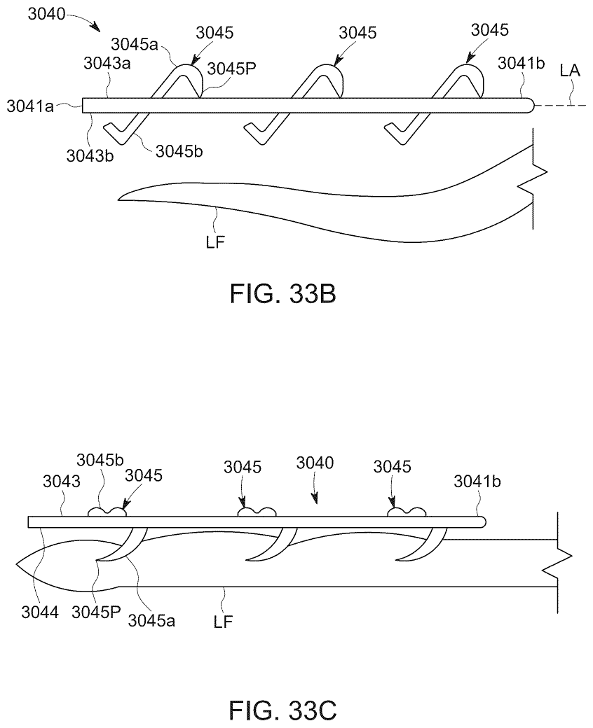

Referring now in addition to FIGS. 33A-33C, which depict one example of a proximal element 3040 according to another embodiment of the present disclosure, which may be incorporated into any of the fixation devices of the disclosure and include any of the characteristics and features of other proximal elements disclosed except as explicitly stated. In one example, proximal element 3040 generally includes an arm 3041 with a first end portion or fixed end 3041a and a second end portion or free end 3041b. Fixed end 3041a may be connected to arm bend feature 153 of gripping device 114 or arm bend feature 260 of gripping device 214 or 214′, for example.

Proximal element 3040 may also include one or more frictional elements 3045 that may each be disposed within a corresponding opening 3046 of arm 3041 and may each be moveably or pivotably connected to arm 3041. For example, each frictional element 3045 may be connected to arm 3041 via one or more hinges 3047 (only one of which is referenced for ease of illustration), which may be a living hinge or a modular hinge, such as a hinge pin, for example. As shown, each frictional element 3045 may include a first portion 3045a and a second portion 3045b with hinge 3047 extending from frictional element 3045 at a location between first and second portions 3045a, 3045b (only one set of portions 2045a, 3045b is referenced for ease of illustration). As such, each frictional element 3045 may be moveable from a first position to a second position and may be biased toward the first position. In the first position, an example of which is shown in FIG. 33B, second portion 3045b of frictional element 3045 may extend downward away from a lower surface 3044 of arm 3041, while first portion 3041a of frictional element 3045 may project upwardly away from an upper surface 3043 of arm 3041. First portion 3045a may include one or more prongs or tips 3045P that may be configured to engage tissue of a leaflet LF and apply atraumatic friction to prevent leaflet LF from sliding relative to proximal element 3040 when engaged therewith. In this regard, first portion 3045a may have a hook shape such that, when frictional element 3045 is in the first position, first portion 3045a extends upwardly away from upper surface 3043, bends downwardly toward upper surface 3043, and terminates with prong 3045P. In the first position, prong 3045P may be positioned either slightly above upper surface or within opening 3046 between upper and lower surface 3043, 3044. This can help shield first portion 3045a from surrounding tissues until leaflet engagement is desired in order to help prevent inadvertently ensnaring other structures of the heart, such as the chordae tendineae CT, for example. In other words, frictional elements 3045 are only exposed when in contact with tissue thereby preventing inadvertent gripper interaction with leaflets and subvalvular apparatus which can lead to tissue damage.

As depicted in FIG. 33C, when proximal element 3045 is lowered onto a proximal side of a leaflet LF, engagement may first occur between first portion 3045a of each frictional element 3045 and leaflet LF. Thus, as proximal element 3045 is lowered, pressure from leaflet LF overcomes the bias of frictional element 3045 which pushes second portion 3045b in a direction toward upper surface 3043 and correspondingly rotates first portion 3045a of frictional element 3045 downwardly in a direction toward bottom surface 3044 and toward leaflet LF. When proximal element 3045 is sufficiently compressed against leaflet LF, frictional elements 3045 are transitioned to the second position in which at least a portion of first portion 3045a, including prong 3045P, is positioned below proximal element 3040 and engages leaflet LF and at least a portion of second portion 3045a of frictional element 3045 may extend from upper surface 3043, as is shown in the illustrated example of the second position. Also, when in the second position, prong 3045P may point in a direction toward fixed end 3041a of arm which may be at least due to the hook shape of frictional element 3045. This helps resist movement of leaflet LF out from between proximal element 3040 and a corresponding distal element, such as distal element 120 or 120′, for example.

Frictional elements 3045 may be made from a shape-memory metal, such as nitinol, for example, which may allow frictional elements 3045 to be laser cut from the same blank of material as arm 3041 and which may provide frictional elements 3045 with a memory to return them to the first position during regrasping. Constructing fictional elements 3045 out of nitinol also allows frictional elements 3045 to be visible under fluoroscopy, which is typically oriented perpendicular to a longitudinal axis LA of proximal element 3040 in a mitral valve procedure during tissue grasping. FIGS. 33C and 33D are illustrative of a fluoroscopic view. Frictional elements may alternatively be made out of a radiopaque material such as platinum-iridium or tantalum or may include a radiopaque marker(s) made out of similar materials, such as at the first and/or second portions of frictional elements 3045. The configuration of frictional elements 3045 described above while in the first and second positions allows the surgeon to observe via fluoroscopy which position each frictional element 3045 is in which may then indicate whether frictional elements 3045 are engaged with tissue or not. In other words, a frictional element 3045 will deploy from its first position to its second position when tissue is positioned between proximal element 3040 and a corresponding distal element and at a location of that particular frictional element 3045, but a frictional element 3045 will not deploy if the tissue is not present at its location. Thus, each proximal element 3040 may have a plurality of frictional elements 3045 arrayed along longitudinal axis LA at predetermined intervals allowing a surgeon to determine, via fluoroscopy, the depth of leaflet insertion by observing which frictional elements 3045 are deployed and which ones are not. It is believed that it is desired to achieve a depth of leaflet insertion beyond 50% of the total length of proximal element arm 3041. Thus, if it is determined that leaflet LF has not reached the desired depth by observing the deployment of frictional elements 3045, then the surgeon may attempt to regrasp leaflet LF until the desired depth is achieved. This can help reduce the incidence of SLDA and implant embolization and help reduce or eliminate the amount of guesswork currently utilized to determine the quality of leaflet capture.

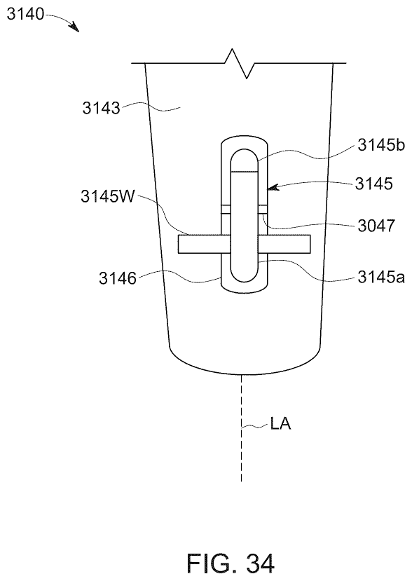

Referring now in addition to FIG. 34, which depicts a proximal element 3140 according to an even further embodiment of the present disclosure, which may be incorporated into any of the fixation devices of the disclosure and include any of the characteristics and features of other proximal elements disclosed except as explicitly stated. In one example, proximal element 3140 is like proximal element 3040 in that it has one or more moveable frictional elements 3145 that are observable under fluoroscopy. However, in this embodiment frictional elements 3145 may be further configured for their respective deployment to be observable under echocardiography. In this regard, each frictional element 3145 may include one or more wings 3145W that project outwardly in a direction transverse to longitudinal axis LA. Such outward projection may be present while frictional element 3145 is in the first position. However, when frictional element 3145 is transitioned to the second position by engagement with a leaflet, wings 3145W may fold inwardly so that they are not visible or are less visible under echocardiography. In other words, wings 3145W may project outwardly from first portion 3145a of frictional element 3145 which may be positioned above an upper surface 3143 of proximal element 3140 when in the first position, but when first portion 3145a moves through opening 3146 during the transition to the second position, upper surface 3143 may engage wings 3145W to fold them inwardly. As such, wings 3145W may be made of a flexible material, such as a shape-memory metal material, such as nitinol, for example. Thus, wings 3145W may return to their outwardly projecting configuration when frictional element 3145 is moved back to the first position, such as during regrasping of tissue, for example.

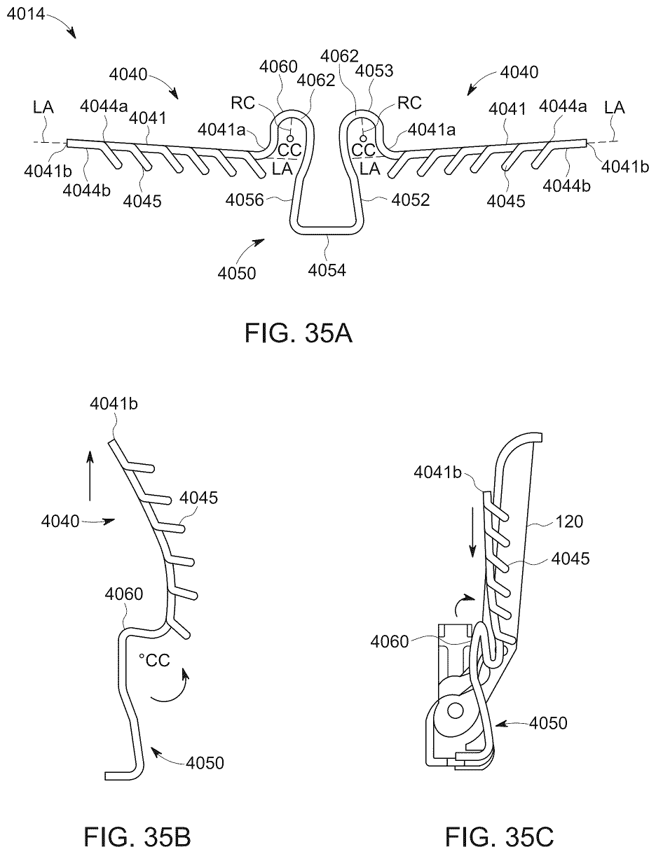

Referring now to FIGS. 35A-35C, which depict a gripping device 4014 according to another embodiment of the present disclosure that may be incorporated into any of the fixation devices described herein. Gripping device 4014 is specifically configured to enhance tissue retention, particularly in circumstances involving shallow leaflet insertion. Additionally, as described below, gripping device 4014 is configured to engage tissue and actively pulls such tissue further into the fixation device after initial engagement.

Gripping device 4014 shares similarities with previously described gripping device 114, except for the differences explicitly detailed below. Components corresponding to those in gripping device 114 use the 4000-series numbering system. For example, base section 4050 corresponds to base section 150, proximal elements 4040 correspond to proximal elements 140, and so forth. Beyond these components, gripping device 4014 also incorporates distinctive arm bend features 4060, which provide enhanced tissue-pulling capability.

As illustrated, each proximal element 4040 takes the form of an elongate arm 4041 with a fixed end 4041a and a free end 4041b. Between these ends are a plurality of frictional elements 4145 that facilitate tissue engagement. Fixed end 4041a of each elongate arm 4041 connects to a corresponding arm bend feature 4060. When gripping device 4014 is in its relaxed (unstressed) configuration, as shown in FIG. 35A, each arm bend feature 4060 curves or bends in a plane parallel to the longitudinal axis LA of its associated elongate arm 4041. As such, each arm bend feature 4060 defines a center of curvature CC and a radius of curvature RC that extends from the arm bend feature 4060 to this center of curvature CC. The radius of curvature may be between 0.02 in. to 0.04 in. In the relaxed state, each arm bend feature 4060 forms an apex 4062 that constitutes the top (proximal) end of the gripping device 4014, while the second member 4054 of base section 4050 forms the bottom (distal) end. In one implementation, each arm bend feature 4060 forms at least a semi-circular shape, with the radius of curvature RC positioned such that the center of curvature CC lies between the longitudinal axis LA of the elongate arm 4041 and the apex 4062. In other words, each arm bend feature 4060 extends upward from the elongate arm 4041 before curving downward toward the base section 4050. In an alternative implementation, the arm bend feature 4060 may form a Z-shaped fold. The rotational span of the arm bend feature 4060 may span approximately 180 degrees (from a 9 o'clock to 3 o'clock position) or may extend up to 225 degrees of rotation, wherein the increased rotational span creates an inward bias of elongate arm 4041 that ensures gripper 4040 compresses inward, thereby pulling tissue into the fixation device as the fixation elements (e.g., fixation elements 120) are closed.

As depicted in FIGS. 35B and 35C, each proximal element 4040 can assume a first grasping configuration and a second grasping configuration. In the first grasping configuration, the proximal element 4040 is tensioned via a proximal element line (e.g., proximal element line 101), causing it to elevate relative to its corresponding fixation element (e.g., fixation element 120) and rotate away from the center of curvature CC of arm bend feature 4060. This tension causes the arm bend feature 4060 to open, unfold, or straighten relative to its relaxed state, effectively elongating the proximal element 4040 and moving the free end 4041b away from the arm bend feature 4060. As shown in FIG. 35C, once tissue is positioned the proximal element 4040 the fixation element 120, releasing this tension allows proximal element 4040 to move toward fixation element 120, enabling the frictional elements 4145 to engage the tissue. As tension continues to decrease, arm bend feature 4060 returns to its bent state under its own bias, pulling the elongate arm 4041 toward the center of the fixation device. This action effectively shortens the proximal element 4040 and draws free end 4041b toward the arm bend feature 4060. The transition from the first to the second grasping configuration creates a translational movement of the frictional elements 4145 along the longitudinal axis LA of the elongate arm 4041 toward the center of the fixation device (e.g., fixation device 112), pulling the engaged tissue deeper between the proximal element 4040 and the fixation element 120. Additionally, the nature of arm bend features is such that elongate arms are disposed lower relative to base section 4050 of gripping device 4014 as compared to elongate arms 141 relative to base section 150 of gripping device 140. Thus, gripping device 4014 is configured to engage tissue at greater than normal fixation device angles (e.g., angles greater than 120 degrees and up to 180 degrees).

Although the subject matter disclosed herein has been described with reference to particular embodiments, it is to be understood that these embodiments are merely illustrative of the principles and applications set forth in this disclosure. It is therefore to be understood that numerous modifications may be made to the exemplary embodiments and that other arrangements may be devised, such as combining one or more features of one embodiment with another embodiment or features from a plurality of embodiments, as an example. Thus, the exemplary embodiments herein are not intended to be exhaustive or to limit the disclosed subject matter to such embodiments.

1. A fixation device comprising:
a first proximal element having a first end portion, a second end portion, a gridded frame disposed between the first end portion and the second end portion, and a plurality of frictional elements, the gridded frame having a plurality of axial struts and a plurality of transverse struts intersecting the axial struts to define a plurality of openings extending through the first proximal element, and at least some of the frictional elements extending from at least some of the transverse struts; and
a first distal element disposed in opposition to the first proximal element and having a first end portion, a second end portion, and a cavity disposed between the first end portion and the second end portion, the cavity being configured to at least partially receive the gridded frame of the first proximal element, the first distal element including a strutted frame disposed between the first end portion and the second end portion of the first distal element, the strutted frame having a pair of inner axial struts and a pair of outer axial struts,
wherein the first proximal element is moveable relative to the first distal element between a first position and a second position, and, when the first proximal element is in the second position, the gridded frame nests within the cavity of the first distal element.

2. A fixation device comprising:
a first proximal element having a first end portion, a second end portion, a gridded frame disposed between the first end portion and the second end portion, and a plurality of frictional elements, the gridded frame having a plurality of axial struts and a plurality of transverse struts intersecting the axial struts to define a plurality of openings extending through the first proximal element, and at least some of the frictional elements extending from at least some of the transverse struts, the first proximal element defining a longitudinal axis extending between the first end portion and the second end portion thereof, and the first proximal element being bent in a transverse plane which extends in a direction transverse to the longitudinal axis; and
a first distal element disposed in opposition to the first proximal element and having a first end portion, a second end portion, and a cavity disposed between the first end portion and the second end portion, the cavity being configured to at least partially receive the gridded frame of the first proximal element,
wherein the first proximal element is moveable relative to the first distal element between a first position and a second position, and, when the first proximal element is in the second position, the gridded frame nests within the cavity of the first distal element.

3. A fixation device comprising:
a first proximal element having a first end portion, a second end portion, a gridded frame disposed between the first end portion and the second end portion, and a plurality of frictional elements, the gridded frame having a plurality of axial struts and a plurality of transverse struts intersecting the axial struts to define a plurality of openings extending through the first proximal element, and at least some of the frictional elements extending from at least some of the transverse struts; and
a first distal element disposed in opposition to the first proximal element and having a first end portion, a second end portion, and a cavity disposed between the first end portion and the second end portion, the cavity being configured to at least partially receive the gridded frame of the first proximal element,
wherein the first proximal element is moveable relative to the first distal element between a first position and a second position, and, when the first proximal element is in the second position, the gridded frame nests within the cavity of the first distal element.

4. The fixation device of claim 3, wherein the first distal element includes a strutted frame disposed between the first end portion and the second end portion of the first distal element, the strutted frame having a pair of inner axial struts and a pair of outer axial struts.

5. The fixation device of claim 4, wherein the plurality of frictional elements includes a pair of inner frictional elements and a pair of outer frictional elements arranged in a row, and when the first proximal element is in the second position, the inner axial struts are disposed between a corresponding pair of inner and outer frictional elements.

6. The fixation device of claim 4, wherein the inner and outer axial struts define a plurality of elongate openings disposed therebetween.

7. The fixation device of claim 4, wherein the outer axial struts each have a proximal surface defining a reference plane, and the inner axial struts are offset from the reference plane in a distal direction.

8. The fixation device of claim 3, wherein the plurality of frictional elements are arranged in a plurality of rows of frictional elements such that at least two rows of the frictional elements each include four frictional elements.

9. The fixation device of claim 8, wherein the first proximal element defines a length extending between the first end portion and the second end portion, the length being bisected by a midline, and the at least two rows of frictional elements each having four frictional elements are disposed at opposite sides of the midline.

10. The fixation device of claim 3, wherein the plurality of frictional elements are arranged in six rows of frictional elements distributed along a length of the first proximal element, the first row of frictional elements being closest to the first end portion of the first proximal element, and the sixth row of frictional elements being closest to the second end portion of the first proximal element, and wherein the first and sixth rows of frictional elements have fewer frictional elements than every row of frictional elements therebetween.

11. The fixation device of claim 4, wherein the first proximal element includes an elongate arm extending from the first end portion to the second end portion, and the gridded frame extends outwardly from the elongate arm such that the elongate arm has a first width, and the gridded frame has a second width greater than the first width.

12. The fixation device of claim 11, wherein the axial struts of the first proximal element define lateral boundaries of the gridded frame, and the transverse struts of the first proximal element extend from the elongate arm to respective axial struts of the first proximal element.

13. The fixation device of claim 11, wherein the outer axial struts of the first distal element at least partially define the cavity and a third width greater than the second width.

14. The fixation device of claim 5, wherein the first proximal element defines a longitudinal axis extending between the first end portion and the second end portion, and the inner and outer frictional elements are angled one of inwardly toward the longitudinal axis and outwardly away from the longitudinal axis such that the outer frictional elements form a first tilt angle and the inner frictional elements form a second tilt angle relative to a distal surface of the first proximal element.

15. The fixation device of claim 14, wherein the first tilt angle is greater than the second tilt angle.

16. The fixation device of claim 3, wherein the first end portion is a fixed end of the first proximal element, and the second end portion is a free end of the first proximal element, the second end portion being bent proximally in a direction away from the frictional elements and defining an opening configured to receive a proximal element line for actuation of the first proximal element between the first and second positions thereof.

17. The fixation device of claim 11, wherein the elongate arm has opposed side edges, the side edges forming at least some of the plurality of frictional elements, and the transverse struts each extend from a corresponding side edge of the elongate arm.

18. The fixation device of claim 3, wherein the plurality of frictional elements include a first frictional element and a second frictional element offset from the first frictional element in a longitudinal direction, the first frictional element being angled relative to a distal surface of the first proximal element by a first angle, and the second frictional element being angled relative to the distal surface of the first proximal element by a second angle, the first angle differing from the second angle.

19. The fixation device of claim 3, wherein the first proximal element defines a longitudinal axis extending between the first end portion and the second end portion thereof, and the first proximal element is bent in a transverse plane which extends in a direction transverse to the longitudinal axis.

20. The fixation device of claim 19, wherein the first proximal element is bent proximally such that a proximal surface thereof is concave in the transverse plane, and a distal surface of the first proximal element is convex in the transverse plane.
